(19) United States
(12) Patent Application Publication
(10) Pub. No.: US 2002/0028193 A1
(43) Pub. Date: Mar. 7, 2002

(54) Recombinant beta2-adrenergic receptor delivery and use in treating airway and vascular diseases

(76) Inventors: Lawrence E. Cornett, Little Rock, AR (US); F. Charles Hiller, Little Rock, AR (US); Stacie M. Jones, Little Rock, AR (US)

Correspondence Address: Richard C. Peet, FOLEY & LARDNER, Washington Harbour, 3000 K Street, N.W., Suite 500, Washington, DC, 20007-5109, US

(21) Appl. No.: 09/783,580

(22) Filed: Feb. 15, 2001

Related U.S. Application Data
(60) Provisional application No. 60/182,502, filed on Feb. 15, 2000.

(51) Int. Cl. A61K 48/00

(52) U.S. Cl. 424/93.21; 514/44; 435/320.1

(57) ABSTRACT

A method of treating a human subject having airway or vascular disease is disclosed that comprises administering to at least one cell type selected from the group consisting of airway epithelial cells, airway smooth muscle cells, blood vessel endothelial cells, and blood vessel smooth muscle cells, a first composition comprising a vector comprising a DNA sequence encoding a β₂AR (p₂-adrenergic receptor) or a mutant thereof, operably linked to a promoter that is functional in at least one of said cells said subject, under conditions whereby the DNA sequence encoding the β₂AR is expressed in at least one of the cells; and optionally administering a second composition comprising at least one β₂-adrenergic agonist into the cells of said subject. A further pharmaceutical composition may be administered comprising a hormone or pharmacological agent that induces the promoter to express the β₂AR in at least one of the target cells. Pharmaceutical compositions comprising the vector comprising a DNA sequence encoding a β₂AR or a mutant thereof, operably linked to a promoter that is functional in at least one of the cells of the subject and kits containing these compositions are also disclosed. An in vitro method of expressing the β₂AR gene in mammalian cells and a method of evaluating the effect of pharmacological compounds on the expression of the β₂AR gene is disclosed.

This application claims priority to U.S. Provisional Patent Application, 60/182,502 filed on Feb. 15, 2000, which is herein incorporated in its entirety by reference.

### BACKGROUND OF THE INVENTION

The present invention relates to a novel treatment of airway and vascular diseases in which dilation of the affected airway or blood vessel would be of benefit to relieve symptoms, in diseases, such as asthma, pulmonary hypertension and systemic hypertension. The present invention provides a method for increasing the β₂-adrenergic receptors (MAR) in the airway epithelial cells and smooth muscle cells and blood vessel endothelial cells and smooth muscle cells resulting in the dilation of the airways and blood vessels, thus relieving symptoms of airway and vascular diseases. The present invention particularly relates to a novel adjunctive treatment of airway and vascular diseases by delivering a first composition comprising a recombinant vector that carries the nucleic acid sequence encoding the native β₂AR or a modified β₂AR, that is delivered into the airways and blood vessels, allowing infection or transduction of at least one type of cell selected from epithelial cells lining the airways, endothelial cells lining the blood vessels and from smooth muscle cells composing the airways and blood vessels.

The present invention more particularly relates to a novel adjunctive treatment of severe asthma in patients who have become hypo-responsive to the bronchodilatory effect of β₂-adrenergic agonists (β₂-agonists) or who may benefit from increasing the bronchodilatory effect of β₂-agonists. The present invention employs a recombinant vector that carries the nucleic acid sequence encoding the native β₂AR or a modification thereof, that is delivered into the airways via bronchoscopy, allowing infection or transduction of at least one type of cell from the epithelial cells lining the airways and from the smooth muscle cells composing the airways and expression of the β₂ARs in these cells. The present invention relates to the delivery of the recombinant vector simultaneously or sequentially with β₂-agonists that are administered to treat acute symptoms by producing rapid bronchodilation.

The present method relates to an in vitro method of expressing the β₂AR gene and evaluating the effect of pharmacological compositions on the expression of β₂ARs in mammalian cells that are transduced with a recombinant vector that carries the nucleic acid sequence encoding the native β₂AR or a modified β₂AR.

Asthma is a collection of symptoms that produces an airway state that causes excessive airway narrowing in response to stimuli that typically do not produce the same effect on the nonasthmatic airway.¹⁵¹ In the past, asthma's etiology was ascribed principally to airway smooth muscle spasm and bronchodilator therapy was the front-line approach for managing asthmatic patients. It is now evident that asthma is a disease of chronic inflammation involving inflammatory cells that release numerous mediators. These mediators initiate airway hyperresponsiveness to various stimuli and lead to the clinical endpoint of bronchoconstriction.⁶ The majority of asthmatic patients display bronchospasm, airway inflammation and mucous plugging. Asthmatics experience recurrent episodes of wheezing, chest tightness, and coughing, particularly at night and in the early morning. In the United States, asthma affects 14 to 15 million individuals and causes more than 5,000 deaths annually. Two general classes of medication are currently available for treatment: long-term control medications (e.g., corticosteroids, cromolyn sodium, methylxanthines, leukotriene modifiers) aimed at obtaining control of the persistent, inflammatory component of asthma and quick relief medications (e.g., short-acting β₂-agonists, anti-cholinergics) used to treat acute symptoms, such as wheezing and shortness of breath.

In the mature airway, the epithelium forms a continuous lining whose cellular composition varies with anatomical location. ^{14,112} From these studies, at least 12 types of airway epithelial cells have been identified, although the proportions of the various cell types vary from species to species. ^{14,112} Within the proximal airway, the epithelial cell layer is thicker and assumes a pseudostratified appearance. ¹⁴ Basal, ciliated, intermediate, and goblet cells are present in the proximal airway and typically there is an absence of Clara cells. In the distal airways, the epithelium becomes thinner. Within the bronchioles, the epithelium is composed of low cuboidal cells and Clara cells become more prevalent. Airway epithelium is generally considered to perform five primary functions:¹¹⁶ 1) acts as a barrier to the diffusion of particles from the airway to underlying lung structures, 2) secretes mucin-like materials, 3) clears the airway of debris through ciliary action, 4) repairs damaged tissue following injury in the airway, and 5) modulates the response of other airway cells particularly to environmental agents that enter the airways. Airway epithelial cells regulate bronchoconstriction,⁴² bronchial vascular responses,⁶⁶ and inflammatory cell recruitment into the airway.^{75,118} Because of easy accessibility, airway epithelium is an attractive target for delivering genetic material to treat both acquired and inherited lung diseases. A prominent example involves the cystic fibrosis transmembrane conductance regulator (CFTR) gene, in which mutations cause the disease cystic fibrosis. ^{1,150}

The bronchodilatory effects of β₂-adrenergic agonists are exerted by stimulating airway smooth muscle relaxation directly and secondarily by causing release of an airway smooth muscle relaxing factor from epithelial cells. FIG. 1 provides a diagram of the β₂AR life cycle following agonist binding to the receptor. Despite their widespread use, β₂-agonists have several shortcomings. For maximum symptom control, frequent dosing is required. This can lead to development of subsensitivity or tolerance, which blunts their effectiveness. Development of tolerance to inhaled β₂-agonists is due to either uncoupling of β₂ARs from downstream effector proteins that produce bronchodilation or the actual loss of β₂ARs from the cell surface.

Although beneficial in the short-term, prolonged use or overuse of β₂-agonists has been associated with reduced β₂AR responsiveness. The phenomenon of reduced β₂AR responsiveness, also known as tachyphylaxis or tolerance, results from a culmination of events, which include desensitization, sequestration, and down-regulation. Desensitization occurs as a consequence of receptor phosphorylation by either protein kinase A (PKA) or a G protein coupled receptor kinases (GRK). Phosphorylation of the receptor causes the receptor to uncouple from downstream effector proteins and also promotes binding of high-affinity arresting proteins, known as arrestins. Binding of arrestins to the β₂AR also targets the receptor for sequestration and down-regulation. Sequestration (or internalization) moves the β₂AR from the cell surface to endosomes located in the cell's interior. Since the native hormones/neurotransmitters epinephrine and norepinephrine cannot cross the cell membrane, internalized receptors are no longer able to trigger a physiological response. Once in endosomes, receptors reach a sorting point in the pathway. The degradation pathway results in a net loss of receptors, also called down-regulation. Internalized β₂ARs can also be recycled back to the cell surface. It is not known what determines whether an internalized β₂AR will be degraded or recycled. Thus, when a severe asthmatic uses his/her β₂-agonist inhaler too many times, levels of β₂ARs decrease in their airways to a point where no matter how much β-agonist they use there are not enough receptors available to bind the drug and produce airway relaxation.

The present invention overcomes this loss of native β₂ARs in asthma patients by the expression of recombinant β₂ARs in airway epithelial and smooth muscle cells to keep β₂AR levels at normal or even at higher than normal levels on the cell surface. A recombinant β₂AR gene, under the control of a very active viral promoter, transcribes high levels of β₂AR MRNA, augmenting the β₂AR mRNA that simultaneously is made by the native β₂AR gene. The niRNA is translated into β₂AR protein and is delivered to the cell membrane at a rate that is faster than the rate at which β₂ARs are removed from the membrane via the desensitization/sequestration/down-regulation pathway.

The β₂AR is a member of a superfamily of membrane assoicated receptors coupled to guanine nucleotide regulatory proteins (G-proteins) and produces its effects by activating intracellular signal transduction pathways.¹³⁶ Results from radioligand assays and receptor autoradiography studies have documented the presence of β₂ARs on a variety of cell types within the mammalian lung.^{8,98} Airway epithelial cells in human,⁷² murine,¹⁰⁶ bovine,¹⁰¹ and rat¹²⁵ lungs express β₂ARs. Several aspects of airway epithelial function are under the control of β₂ARs. β₂-agonists increase bronchial epithelial chloride and mucus secretion, and increase ciliary activity.⁹⁹ The effects of inhaled β₂-agonists on mucociliary clearance in humans seem to be variable. In one study, the β₂-agonist, procaterol, had no effect in both controls and in asthmatic individuals.⁶⁸ However, inhaled terbutaline increases mucociliary clearance in both controls and in patients with asthma.⁹³ Results from several studies have suggested that the relaxation of airway smooth muscle that occurs in response to β₂-agonists is reduced if the bronchial epithelium is removed.^{42,155} This finding could be explained by the release of a smooth muscle relaxing factor from bronchial epithelial cells in response to stimulation with β₂-agonists. This factor, designated epithelium-derived relaxant factor (EpDRF),^{42,155} could either act directly on smooth muscle to cause relaxation or alternatively enhance the effect of β₂-agonists on bronchial smooth muscle (See a schematic of the proposed mechanism in FIG. 2). β₂-agonists and glucocorticoids are the two most effective treatments available for asthma therapy and frequently are used in combination. Glucocorticoids are used principally because of their anti-inflammatory properties,⁷ although additional beneficial effects of glucocorticoids in the asthmatic lung have been observed. ¹³⁵ Synthetic glucocorticoids are efficacious for treating asthma and other diseases with associated inflammatory processes because they mimic glucocorticoids produced endogenously by the adrenal cortex. The cellular and molecular mechanisms of action of the glucocorticoids have been extensively studied. ^{135 154} The current model of glucocorticoid action postulates intracellular glucocorticoid receptors, which in the absence of ligand, are complexed with heat shock proteins (hsp90, hsp56, hsp70 and an acidic 23 kD protein) (See FIG. 3). Glucocorticoids are found in the blood bound to transcortin, albumin, and other serum proteins. Free glucocorticoids enter target cells, by still unidentified mechanisms ^{49,152} and bind to glucocorticoid receptors causing dissociation of the associated heat shock proteins. Association of hsp90 with glucocorticoid receptors appears to maintain the hormone binding domain in its high affinity conformation.¹¹³ The functional roles of the other associated heat shock proteins are not as well understood, but may include trafficking of the receptor within the cell.¹¹³ The glucocorticoid receptor is a member of a superfamily of transcriptional regulators that include receptors for estrogens, progesterones, androgens, vitamin D, thyroid hormones, and retinoic acid.⁸³ Members of the superfamily share a similar structure with functional domains for binding of hormone, binding to DNA, and transcriptional activation. Hormone-receptor complex translocates from the cytoplasm to the nucleus. Activated glucocorticoid receptor with hormone bound has an increased affinity for binding to specific DNA sites termed glucocorticoid response elements (GRE) found within glucocorticoid reponsive genes. GREs can either be simple or composite.⁴⁸ Most simple GREs consist of two half-site hexamers separated by three nucleotides with resemblance to the consensus sequence GTCACAnnnTGTTCT. Association of glucocorticoid receptor, typically as a homodimer, to simple GREs results in enhanced transcription of the target gene. A second type of DNA sequence that binds glucocorticoid receptors, termed composite GREs, has been found in certain glucocorticoid-responsive genes.³¹ At composite GREs, the hormone receptor complex interacts with both specific DNA sequences and other transcription factors to regulate transcription.^{31,47,91} The first demonstrated composite GRE was shown to have binding sites for both the glucocorticoid receptor and activating protein-1 (AP-1).³¹ AP-1 is a dimer of the oncogene products c-fos and c-jun. Since glucocorticoid receptors are expressed in many cell types, composite GREs may explain how signal specificity can be achieved in a system with an apparent common fmal pathway.⁴⁸

For many G-protein coupled receptors, modulation of receptor number is an established mechanism controlling responsiveness to hormones and neurotransmitters. Heterologous regulation of β₂AR levels by glucocorticoids is a physiologically important example of such control.²³ Numerous in vitro and in vivo studies in a variety of cell types have shown that β₂AR levels and β-agonist-stimulated adenylyl cyclase activity are increased by glucocorticoids.^{19,28,46,104,140,157} Glucocorticoids increase EAR levels in the lung of several species including rat, rabbit and human. ^{19 84,87} The increase in β₂AR number results from an increase in rate of new receptor synthesis,¹⁰⁴ which is preceded by increased steady-state levels of β₂AR MRNA ^{23,28,58,59,81,90,140} and increased transcription rate of the β₂AR gene.^{23,59,81} The increase in EAR density induced by glucocorticoids is blocked by the transcriptional inhibitor actinomycin D.⁹⁵ Taken together, these findings suggest that enhanced β₂AR gene transcription is a principal mechanism underlying glucocorticoid mediated increases in β₂AR levels. The β₂AR genes from several mammalian species contain GRE-like sequences in both coding and non-coding regions.^{16,39,70,73,88} Although indirect evidence suggests that the 5′-noncoding region of the β₂AR gene is involved,⁸² data discussed below identifies specific genetic elements responsible for the functional effect of glucocorticoids on β₂AR gene transcription.

Since the development of β₂-adrenergic selective drugs and metered-dose inhaler delivery systems, agents that target the β₂ARs have become the most commonly prescribed medications for asthma.³⁴ The principal beneficial effects of β₂-agonists are on bronchomotor tone and airway patency. Agonist stimulation of β₂ARs in airway smooth muscle inhibits contractile processes, resulting in bronchodilation.¹¹⁵ This is an important property of β₂ARs because several bronchoconstricters (histamine, bradykinin, acetylcholine, LTD₄, and PGD₂) are elevated in the asthmatic lung.⁵

β₂-agonists are effective bronchodilators in large and small airways.⁹⁷ β₂AR-mediated processes in airway epithelial cells (e.g., mucous clearance, production of bronchoactive factors) may also either directly or indirectly affect the contractile state of airway smooth muscle,⁹² β₂-agonists have been shown to decrease mediator release from basophils and mast cells.^{22,109} Administration of β₂-agonists reduces vascular leakage caused by inflammatory mediators including histamine, platelet activating factor, and certain prostaglandins.^{105,111} Finally, β₂ARs are expressed on the surface of potential inflammatory cells, including eosinophils, alveolar macrophages, lymphocytes and polymorphonuclear leukocytes ^{24,33,78,156} While the role of the β₂AR on proinflammatory cells in the asthmatic lung is currently unclear, some physiological effects have been reported. β₂-agonist treatment in vitro is associated with decreased proliferation of human T lymphocytes in response to mitogenic stimuli²⁴ and inhibition of lysosomal enzyme release from granulocytes.⁶⁵

Despite their well-documented clinical efficacy, regular use of β₂-agonists and glucocorticoids can lead to several clinical problems. Doubts concerning the safety of β₂-agonists arose from epidemiologic studies conducted in New Zealand^{25,55,110} and Canada¹³³ that demonstrated an association between the regular use of β₂-agonists, particularly fenoterol, and the risk of dying from bronchial asthma. The conclusions drawn from these studies are controversial and do not necessarily prove a cause-effect relationship since patients with more severe asthma, and therefore a higher risk of fatal attacks, are more likely to use β₂-agonists more frequently or at higher doses.⁵ A balanced review on the safety of β₂-agonists also can be found.¹⁴¹ Overuse of inhaled β₂-agonists may also increase asthma morbidity. Separate studies have shown that regular β₂-agonist use can be associated with poorer control of symptoms and increased airway hyper-responsiveness compared to asthmatics who use the same medications “as needed”.^{128 142} These observations are not surprising since it is well-known that β₂ARs are prone to the process of desensitization and down-regulation resulting in loss of receptor function following prolonged β₂-agonist exposure.^{57,74} The present invention circumvents the adverse effects associated with overuse of β-agonists.

Because all cells express a common glucocorticoid receptor, every cell and organ system can be affected by administration of exogeneous glucocorticoids. Numerous potential adverse effects that can arise from systemic glucocorticoid use,¹³² and include effects that can occur immediately (e.g., hypokalemia and hyperglycemia), those that develop over a longer period of time (e.g., osteoporosis and cataracts), and those that are limited to children (e.g., growth suppression). Chronic glucocorticoid use results in development of posterior subcapsular cataracts with a prevalence of up to 29% in adults and children.^{120, 145} Patients on >7.5 mg/day prednisone for longer than 6 months are at risk for developing osteoporosis.⁷⁹ Inhibition of linear growth in children has been observed with regular daily therapy, frequent short course therapy and high-dose alternate day therapy with glucocorticoids. ^{10,96,129} Inhaled glucocorticoids, used as long-term management by many asthmatics, generally display fewer risks than oral glucocortoids.⁵³ At doses up to 800 μg/day, few clinically important adverse effects are observed with oral glucocorticoids; however, with long-term use of doses greater than 800 μg/day, osteoporosis, suppressed linear growth in children, adrenal-pituitary axis suppression, and cataracts are the most likely adverse effects to develop.^{15,53,143} Although inhaled corticosteroids have significantly less potential for causing adverse effects compared to oral systemic corticosteroids, current recommendations are to use the lowest possible dose to maintain control of symptoms.⁴⁰ Some of the beneficial effects of glucocorticoids are the result of increased β₂AR numbers. The present invention provides a method whereby β₂AR overexpression in airway epithelial cells and smooth muscle cells leads to decreased glucocorticoid requirements in severe asthmatics.

Shortcomings in safety and persistently delivering effective quantities of specific proteins to patients using recombinantly produced proteins has led to the development of gene therapy methods for delivering sustained levels of specific proteins into patients. Gene therapy is defined as the insertion into a patient of DNA that codes for either normal or altered genes,in order to correct a genetic or acquired disorder. The normal or altered gene that is inserted corrects the disorder via production in the patient of either missing, defective, or insufficient gene products. The DNA may be introduced by known cell transfection methods, but viral-mediated gene delivery methods, such as retrovirus, adenovirus, herpes virus, pox virus, and adeno-associated virus (AAV) are used in more than 95% of gene therapy trials conducted. ¹⁵⁹

Gene therapy techniques utilize various vehicles for gene transfer and Table 1 of Kay et al.¹⁶⁰ disclose retroviruses, adenoviruses and AAV as viral vehicles and liposomes as nonviral vehicles. Each one of these vehicles has limitations in gene therapy applications (discussed in Stone et al.¹⁶¹). Nonviral vechicles, such as liposomes presently lack target cell specificity. Widespread expression of transgenes can have deleterious outcomes. The disadvantages of retroviral vectors include random insertion into the host genome, inactivation by human complement, inability to transduce non-dividing cells, and possible decreased transgene expression over time. Additionally and very importantly, the retroviruses and retroviral vectors have been banned for use in gene therapy by the Food and Drug Administration. The principal drawback of adenoviruses is a significant host immune response against the viral vector, vector-encoding proteins, and the cells expressing these proteins. This leads to inflanunation and elimination of transduced cells by the immune system, requiring frequent re-administration of the transgene. Because adenovirus does not incorporate into the host genome, duration of transgene expression is limited. AAV possesses a number of features not possessed by the other viruses, such as: wide host range, ability to infect different species, no known association with any human or animal disease; does not appear to alter the biological properties of the host cell when it integrates, its stability over a wide range of physical and chemical conditions, its small size, and less complicated epitopes presented to a patient than adenoviruses. For all of these reasons, a more suitable choice of a vector for transducing epithelial cells, is the AAV vector as originally suggested by Hermonat et al.⁶²

The AAV genome is a linear, single-stranded DNA molecule containing 4681 nucleotide and an internal non-repeating genome flanked on each end by inverted terminal repeats (ITRs). The ITRs are approximately 145 base pairs in length and have multiple functions. The internal non-repeated portion of the genome includes two large open reading frames, known as the AAV replication (rep) and capsid (cap) genes, code for viral proteins that allow the virus to replicate and package the viral genome into a virion.

AAV is a helper-dependent virus that requires co-infections with a helper virus, such as adenovirus, herpesvirus or vaccinia) in order to form AAV virions. AAV has been engineered to contain heterologous genes by deleting the internal non-repeating portion of the AAV genome (i.e., the rep and cap genes) and inserting the heterologous gene linked to a promoter between the ITRs. The ITRs are the only viral elements necessary for efficient encapsidation and integration of the viral genome of the host cell. ¹⁶² The AAV genome stably integrates into a specific site in human chromosome 19¹⁶³ and AAV is able to transduce both mitotic and post-mitotic cells.¹⁶⁴ In order to prepare AAV a helper virus (usually adenovirus) is required. Although high titer AAV preparations result, the preparations are contaminated with helper virus that must be removed by tedious purification steps. Alternative strategies to prepare recombinant AAV have been developed¹⁶⁵ (See also U.S. Pat. No. 6,004,797, U.S. Pat. No. 6,001,650, U.S. Pat. No. 5,945,335) that produce high AAV titers with no helper virus contamination.

In regard to the usefulness of gene therapy methods for the treatment of asthma, a number of research groups have looked at new treatments for asthma. Demoly et al. (1997) ¹⁶⁶ considered the possibility of a gene therapy based strategy for the treatment of asthma. This publication admits that in the treatment of asthma, there are no deficient genes to replace and no protein is truly repressed but rather there is a hypersecretion. These authors consider blocking the expression of an inflammatory or immunoregulatory protein but admit that sophisticated gene therapy techniques would be required. Further, a publication by Rogers et al. (1998)¹⁶⁷, considers gene therapy as a new approach to treat lung disease because the lung provides an accessible target through the airways or the vasculature. This publication considers the lung diseases of α₁-antitrypsin deficiency and cystic fibrosis as good candidates for gene therapy because the genetic defect in each disease is well characterized. This publication also considers the different vectors and the advantages and disadvantages of using them.

McGraw et al. reported at the 1998 ALA/ATS International Conference⁸⁹ and in a later publication¹⁶⁸ that their group had generated transgenic mice lines using the human surfactant protein C promoter and the rat CC10 promoter to overexpress the human β₂AR in distal alveolar epithelium (type II cells) and bronchial tracheal epithelium (Clara cells). Epithelial cell β₂AR overexpression was verified by receptor autoradiography. These transgenic mice lines may be useful in selectively isolating the effect of proximal and distal epithelial cell β₂ARs upon airway physiology and pathophysiology. In 1999, McGraw et al. ¹⁶⁹ reported that transgenic mice expressed β₂ARs in airway smooth muscle using a mouse smooth muscle α-actin promoter as compared to normal mice.

Another group looked at the overexpression of β₂AR in adenoviral transduced cultured adult rabbit ventricular myocytes,¹⁷⁰ and concluded that recombinant adenoviral gene transfer of EAR or an inhibitor of β-adrenergic receptor kinase (βARK)-mediated desensitization can potentiate β-adrenergic signaling. Recently another publication by some of the members of the previous group, delivered adenoviral transgenes including the human β₂AR gene to the myocardium of rabbits using catheter-mediated delivery. This study demonstrated global myocardial in vivo gene delivery and that genetic manipulation of β₂AR density can result in enhanced cardiac performance as compared to control rabbits.

The human β₂AR gene is known and sequenced. The first report of cloning and sequencing the human β₂-AR was reported in. ^{171, 73}

As discussed above, a significant problem associated with prolonged treatment of asthmatics with β₂-agonist treatment is desensitization or tolerance. The loss of clinical responsiveness to β₂-agonists has potentially serious outcomes including increased mortality, poor baseline asthma control, escalating medication usage, and increased cost of care. The present invention provides a recombinant vector and a method of using this vector to provide additional β₂ARs to airway epithelial cells and smooth muscle cells, but particularly airway epithelial cells, to provide increased levels of β₂ARs in these cells. The increased levels of β₂ARs alone or with adjunct β₂-agonists or controlled β₂AR expression by endogenous or administered inducers of the promoter operably linked to the β₂AR gene provide a method of treating airway diseases, such as asthma. The present method also is useful for providing treatment for other diseases that can benefit from increased levels of β₂ARs, such as vascular diseases.

### SUMMARY OF THE INVENTION

The present invention relates to vectors comprising a DNA sequence encoding a β₂AR operably linked to a promoter that is functional in at least one cell type of the airways and blood vessels of a human subject. In a preferred embodiment, the vectors are adeno-associated virus based. Adeno-associated virus has a natural tropism for airway epithelia. So these adeno-associated virus based vectors are particularly preferred for respiratory gene therapy applications. The present invention is directed to cells containing the vector.

The present invention is further directed to a method for providing a β₂AR to airway epithelial cells, airway smooth muscle cells, blood vessel endothelial cells, blood vessel smooth muscle cells or a combination thereof, of a human subject comprising administering to at least one of these enumerated cell types, a first composition comprising a vector comprising a DNA sequence encoding a β₂AR operably linked to a promoter that is functional in at least one of the cells, under conditions whereby the DNA sequence encoding said β₂AR is expressed in at least one of these cells. In a further embodiment, the DNA sequence encodes a β₂AR that is modified in its function as compared to the native β₂AR.

The present invention further is directed to an adjunct therapy for treating a human subject having airway or vascular disease comprising administering to at least one cell type selected from the group consisting of airway epithelial cells, airway smooth muscle cells, blood vessel endothelial cells, and blood vessel smooth muscle cells of the human subject, a first composition comprising a vector comprising a DNA sequence encoding a β₂AR operably linked to a promoter that is functional in at least one of these types of cells of the subject, under conditions whereby the DNA sequence encoding the β₂AR is expressed in at least one of these types of cells; and administering a second composition comprising at least one β₂-agonist to at least one cell type of the subject.

The present invention additionally is directed to administering simultaneously or after the administeration of a vector comprising a DNA sequence encoding a β₂AR operably linked to a promoter that is functional in at least one of the types of cells of the subject enumberated above, a hormone or other pharmacological agent that induces the promoter to express β₂ARs.

The present invention is further directed to pharmaceutical compositions containing the vector comprising a DNA sequence encoding a β₂AR operably linked to a promoter that is functional in cells of the airway epithelium and smooth muscle cells and blood vessel endothelium and smooth muscles.

The present invention additionally is directed to a kit that contains in separate containers at least one pharmaceutical composition comprising the vector comprising a DNA sequence encoding a β₂AR operably linked to a promoter that is functional in cells of the airway epithelium and smooth muscles and blood vessel endothelium and smooth muscles, at least one additional pharmaceutical composition comprising a β₂-agonist, and optionally at least one pharmacological agent that induces the promoter to express β₂ARs in the target cells. The pharmaceutical compositions are in a formulation suitable for aerosol delivery or intravenous delivery.

The present method further is directed to an in vitro method of expressing the β₂AR gene and evaluating the effect of pharmacological compositions on the expression of β₂ARs in mammalian cells that are transduced with a recombinant vector that carries the nucleic acid sequence encoding the native β₂AR or a modified β₂AR.

### BRIEF DESCRIPTION OF THE DRAWINGS

FIG. 1 depicts the β₂-AR life cycle following agonist binding to the receptor.

FIG. 2 depicts a representation of the a mechanism by which relaxation of airway smooth muscle is induced by β₂-agonists.

FIG. 3 depicts transcriptional regulation of the β₂-AR gene expression by glucocorticoids.

FIG. 4 depicts AAV vectors and complementor genomic structure. The four phenotypic regions of AAV are shown. The rep region encodes products required for AAV DNA replication. The lip and cap regions encode the virion capsid proteins. The terminal repeats (tr) are required in cis for AAV replication, packaging and integration into host DNA. P5 is the AAV promoter. d13-94 and d16-95 are used as backbones for insertion of the β₂AR and neomycin genes. d16-95/GFP/Neo and d16-95/LacZ/Neo are used in experiments to optimize conditions for expression of viral vectors and localization of transduced cells in the rat lung. ins96-λ-M are used to package recombinant AAV vectors.

FIG. 5 shows the transduction of SPOC1 cells by AAV-GFP. Panels A and B, SPOC1 cells infected with recombinant AAV (d16-95/GFP/Neo) observed using fluorescence microscopy, Panels C and D, the same cells observed using interference contrast microscopy.

FIG. 6 depicts the genomic structure of recombinant AAV vectors. For reference, the genomic structure of wildtype AAV is shown at the top. Descriptions of each vector can be found in the text. β₂AR(tag) refers to a cassette that contains the β₂AR coding region with an epitope (YPYDVPDYA) added at the amino terminus of the receptor open reading frame. The epitope tag does not alter β₂AR function¹⁴⁷ and can be detected with a specific antibody.¹⁰⁰

FIG. 7 shows the effect of methacholine on airway resistance in anesthesized Brown-Norway rats. Panels A and B, Animals were injected ip with 0.9% NaCl and two weeks later exposed to nebulized 0.9% NaCl for 30 min. Panels C and D, animals were injected ip with 1 mg ovalbumin/200 mg aluminum hydroxide in 0.9% NaCl and two weeks later exposed to nebulized ovalbumin (1 mg/mI). Animals were anesthesized with urethane and placed on ventilators so that airflow (Panels B and D) was constant. Methacholine was administered to the animal in nebulized form. The concentration of methacholine in the solution was 1 mg/ml.

FIG. 8 depicts putative glucocorticoid response elements (GRE) in the rat β₂-AR gene.

FIG. 8A provides a schematic representation of the β₂AR gene. GREs are number and approximate locations are shown.

FIG. 8B shows the exact locations (+1 is the start of transcription) of the putative GREs. The third column shows the nucleotide sequence of each GRE compared to the MMTV consensus GRE. Underlined nucleotides match consensus. The number of matching mucleotides compared to the consensus GRE are shown in column 4.

FIG. 9 shows the expression of β₂AR-luciferase fusion genes in HepG2 cells incubated in the absence or presence of 0.1 μM dexamethasone for 8 hours. HepG2 cells were transiently transfected with pRShGRα, pRSVβ-ga1, and either pβ₂AR(−62/+126), pβ₂AR(−152/+126), pβ₂AR(−643/+126), pβ₂AR(−1115/+126), pβ₂AR(−2552/+126), pβ₂AR(−3129/+126), or N-600 prATLUC. Transfected cells were incubated for 48 hours prior to addition of dexamethasone. Luciferase activity for each construct is expressed relative to that obtained with pβ₂AR(−62/+126) in the absence of dexamethasone. Results are expressed as the mean ±S.E.M. from eight independent experiments, each conducted in triplicate. *Significantly (p<0.02) different by Student's t-test.

FIG. 10 shows oligonucleotides used in electrophoretic mobility shift assays and luciferase assays with pT81LUC. Both sense and antisense oligonucleotides were made by Bio-Synthesis (Lewisville, Tex.). Only the sense oligonucleotides for each complementary pair are shown. Bold nucleotides represent β₂AR gene sequence and italicized nucleotides represent restriction enzyme sites added to facilitate cloning into pT81LUC. Underlined nucleotides represent putative core GRE elements. The mutated nucleotide in the putative core GRE element in GRE₅ is underlined and italicized.

FIG. 11 shows the effect of a single point mutation in GRE₅ on dexamethasone inducibility of a β₂AR-luciferase fusion gene. Human HepG2 cells were transiently transfected with pRShGRα, pRSVβ-gal, and either pβ₂AR(−3129/+126) or pβ₂ARm1 (−3129/+126) as described in the specification. After transfection, the cells were incubated for 8 hours in either the absence or presence of 0.1 μM dexamethasone and cells were harvested. Values are means±S.E. of data from five independent experiments, each performed in triplicate. Asterisks indicate a significant (p<0.05) difference in luciferase activity from untreated and dexamethasone-treated cells as determined by Student's t-test.

FIG. 12 shows dexamethasone induction of a heterologous thymidine kinase promoter fused to various glucocorticoid response elements. Plasmid constructs with the luciferase gene under the control of the tk promoter and various putative GREs (see FIG. 8 for sequences) were tested for luciferase activity after transfection into HepG2 cells that were co-transfected with pRShGRα. Transfected cells were incubated for 48 hours prior to addition of 0.1 μM dexamethasone. Results are expressed as the mean∓S.E. from four independent experiments, each conducted in triplicate. *Significantly (p<0.01) different by Student's t-test.

FIG. 13 shows characterization of HepG2 cell nuclear proteins that interact with GRE₅ by electrophoretic mobility shift assays. HepG2 cell nuclear extract (6 μg) was incubated with radiolabeled GRE₅ in the presence of increasing concentrations (10-, 50-, 100-, 250-, or 500-fold molar excess) of the indicated double stranded oligonucletide.

FIG. 14 shows the specificity of the interaction between GRE₅ and human recombinant glucocorticoid receptor by electrophoretic mobility shift assays. Human recombinant glucocorticoid receptor was incubated with radiolabeled GRE₅ in the presence of increasing concentrations (25-, 100-, or 250-fold molar excess) of either unlabeled GRE₅, m1GRE₁ (see FIG. 10 for sequences).

FIG. 15 shows [¹²⁵I] CYP binding to SPOC1 cell membranes: Saturation analysis. SPOCI cell membranes were incubated at 37° C. for 2 hour with increasing concentrations of [¹²⁵I] CYP. Nonspecific binding was defined with 0.1 μM (−)-propranolol. Scatchard analysis of specific binding (open circles) demonstrated that [¹²⁵I]CYP binding was saturable and displayed high affinity. Inset: Direct plot demonstrating total binding (closed circles), non-specific binding (open triangles) and specific binding (open circles).

FIG. 16 shows cyclic AMP accumulation in SPOC1 cells in response to isoproterenol in the presence and absence of a phosphodiesterase inhibitor. SPOC1 cells were treated for 10 min at 37° C. with either vehicle, 100 μM IBMX, 10 μM isoproterenol, or 100 μM IBMX and 10 μM isoproterenol in combination. Cyclic AMP accumulation was measured by radioimmunoassay. Significant differences were determined by one way ANOVA and Neuman-Keuls test.

FIG. 17 depicts expression of β₂AR-luciferase fusion genes in SPOC1 cells. SPOC1 cells were transiently transfected with 0.2 μg pRLV-SV40, 1.6 μg pGEM7Zf(−), and 0.2 μg of either pβ₂AR(−283/−95) or pβ₂AR(-3349/-95). Cells were incubated for 8 hours in either the presence or absence of deamethasone. Luciferase activities are expressed relative to that of pβ₂AR(−283/−95) in the absence of dexamethasone. Results are expressed as the mean±S.E.M. from five independent experiments each performed in triplicate. *Significantly (p<0.01) different by Student's t-test.

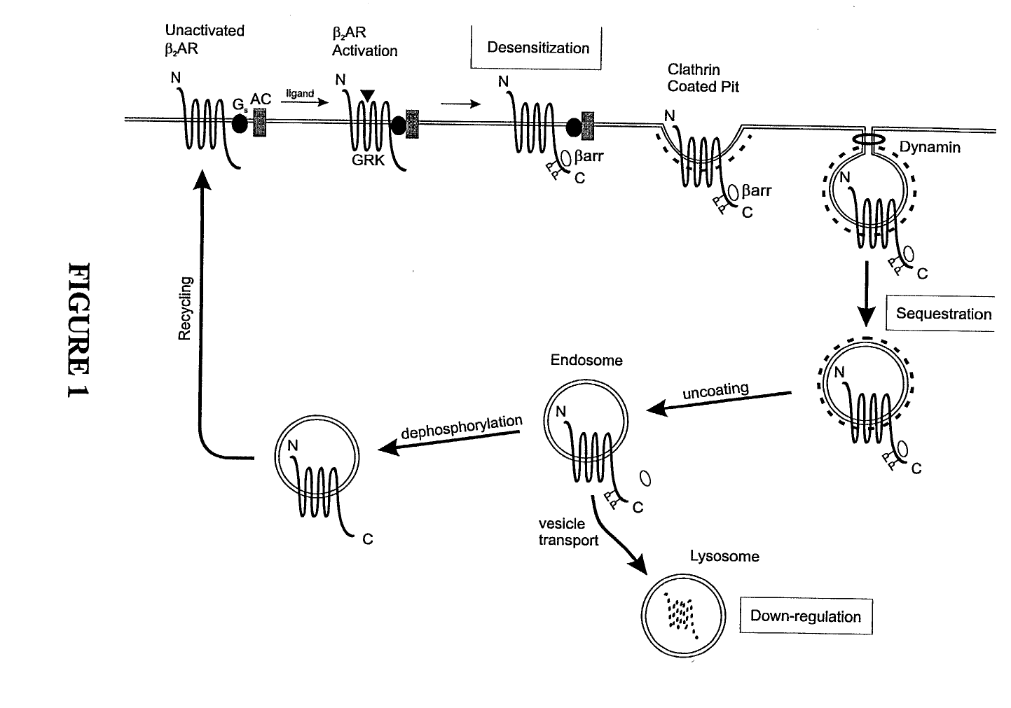

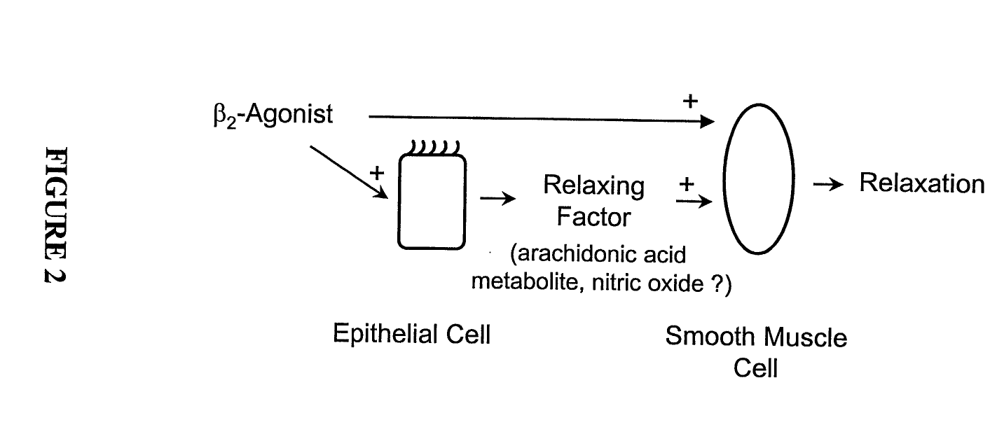

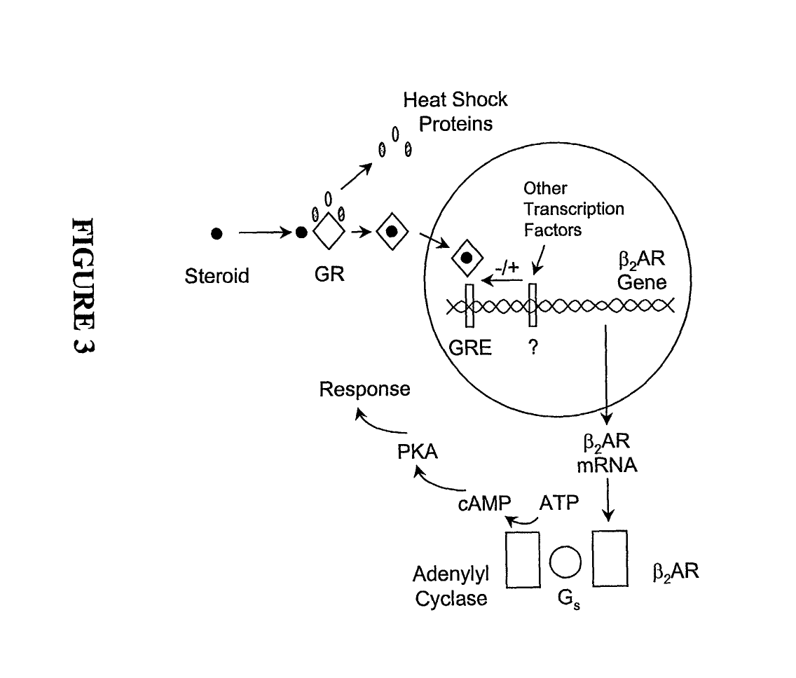

### DETAILED DESCRIPTION OF THE PREFERRED EMBODIMENT

The present invention is directed to a novel approach for adjunctive treatment of patients having airway and/or vascular diseases, in which dilation of the affected airways or blood vessels would be of benefit to relieve symptoms, in diseases, such as asthma, and pulmonary or systemic hypertension. The present treatment utilizes gene therapy to dilate the airways and blood vessels by providing increased levels of β₂ARs for endogenous hormones, such as epinephrine or norepinephrine. Optionally the β₂AR gene containing try vector is administered in conjunction with β₂-agonists to enhance treatment by providing increased levels of β₂ARs for the β₂-agonist to bind. More specifically, a vector comprising a β₂AR gene under the control of regulatory sequences that express the gene in the airway epithelial cells and/or airway smooth muscle cells, is delivered to the patient's airway, and optionally, either simultaneously or sequentially, a β₂-adrenergic agonist is delivered to the patient's airway. Similarly, a vector comprising a β₂-AR gene under the control of regulatory sequences that express the gene in the blood vessel endothelial cells and/or blood vessel smooth muscle cells, is delivered to the patient's blood stream, and optionally, either simultaneously or sequentially, a β₂-agonist is delivered to the patient's blood stream.

In a preferred embodiment, the present invention is directed to a novel adjunctive treatment for patients or human subjects with severe asthma who become hypo-responsive to the bronchodilatory effects of β₂-agonists. The treatment method of the present invention comprises administering a vector comprising the β₂AR gene, and optionally administering a β₂-agonist into the airways of the patient. The vector and the β₂-agonist can be administered simultaneously or sequentially, resulting in the airway epithelial cells being transduced by the vector with the resulting infection of airway epithelial cells lining the patients' airways, and also the infection of the smooth muscle composing the epithelial cell boundary, and possibly even entry into the bloodstream. The β₂ARs are expressed in the transduced cells, providing additional β₂ARs to which the administered β₂-agonist binds. By the present method of treatment, the airway smooth muscles are relaxed and airway dilation is achieved.

The present invention differs from standard gene therapy approach for treating diseases in that it is not utilized to replace a defective protein with a version of the protein that functions properly. The present invention provides increased numbers of β₂ARs on the surface of the epithelial cells, in addition to the native β₂ARs already present. In the treatment of asthma, there is no evidence that the β₂AR is defective, and that this is the cause of the asthmatic condition. Yet, the β₂AR is an important therapeutic target in the treatment of asthma.

The principal clinical benefits anticipated in asthmatic patients receiving β₂AR gene therapy are: 1) increased sensitivity to the airway relaxing effects of circulating epinephrine and norepinephrine, and inhaled β₂-agonists resulting in decreased dependency upon β₂-agonist medications; and 2) decreased susceptibility to the development of subsensitivity or tolerance to inhaled β₂-agonists. The asthma treatment of the present invention is effective in decreasing the incidence of worsening asthma control and mortality that is associated with frequent use of β₂-agonists in some patients who are frequent users of β₂-agonists and suffer from the negative effects of tachyphylaxis.

The present invention also is directed to the use of an inducible β₂-AR gene that is targeted to airway epithelial and/or smooth muscle cells to dilate the airways by increasing the number of β₂-ARs, but also is useful to improve airway responsiveness to β₂-agonists. The use of gene therapy for the management of individuals, whose asthma is poorly controlled by current conventional treatment methods, provides a useful and innovative treatment to overcome desensitization to β₂-agonists. The present invention is built upon the kinetic relationship between β₂-agonists and β₂-ARs to produce beneficial physiological responses (e.g., bronchodilation).

The present invention discloses a new therapy that provides a means to increase β₂-adrenergic responsiveness by increasing levels of β₂AR in airway epithelial cells and/or airway smooth muscle cells. This is accomplished in a step-wise fashion, that in addition to creating a novel approach to treating asthma, advances basic knowledge in research in lung biology. Although the airway smooth muscle possibly represents a more physiologically significant target for gene therapy involving the β₂AR, the present invention focuses on airway epithelium for several reasons. First, there is evidence that part of the airway relaxing effects of β₂-agonists are mediated via interactions with epithelial cell β₂ARs. Second, airway epithelial cells are easily targeted using vectors derived from either adenoviruses or adeno-associated viruses (AAV). Moreover, the feasibility of transducing epithelial cells with recombinant viruses carrying a mammalian gene and achieving expression of a functional protein has been demonstrated by numerous cystic fibrosis laboratories working with the CFTR protein.²⁶ This is a procedure that has been used in humans. Third, the present invention provides a practical procedure for specifically targeting and/or expressing a recombinant gene in airway epithelial cells and smooth muscle cells that can be safely used in humans.

The vector and the β₂-agonist is administered simultaneously or sequentially, but preferably the vector comprising the β₂AR gene is administered, and the airway epithelial cells and/or smooth muscle cells are transduced, resulting in the infection of airway epithelial cells and/or smooth muscle cells. The vector containing the β₂AR gene is administered to the subset of asthmatic patients, who are difficult to manage with traditional therapies (e.g., the ones that end up in the emergency room). The recombinant β₂AR vector is administered in aerosolized form, in the same manner that β₂-agonists and glucocorticoids are taken by asthmatics.

Vehicles for gene transfer to cells may be selected from retroviruses, adenoviruses, AAV or nonviral vehicles. But the present invention prefers AAV vectors as the vehicle for transduction. Examples of U.S. patents, disclosing viral transduction of genes using AAV based vectors are U.S. Pat. No. 5,670,488; U.S. Pat. No. 5,139,941 and U.S. Pat. No. 5,252,479. An example of non-viral transfection of lungs is disclosed in U.S. Pat. No. 6,022,737.

After a sufficient period to allow for the transduction of the epithelial cells and the expression of the β₂ARs in the epithelial cells, for example for approximately 1 to 2 days, the β₂-agonist is optionally administered. During this period of time, there is episomal expression that is transient. Long-term expression occurs as the recombinant virus integrates into the host genome. This occurs in a specific location on human chromosome 19. Increased levels of β₂AR are present in infected cells over the course of the cell's lifetime, approximately 180 days in human airways. During that time period, individuals administered the vector containing the β₂AR gene are hyperresponsive to aerosolized β-adrenergic agonist, compared to individuals not given the vector. Eventually, the beneficial effect diminishes as the airway epithelial cells slough off, divide, and otherwise disappear from the airway. Thus, it is necessary to reinfect a human subject or patient with the recombinant β₂AR vector if control of the patient's asthma becomes difficult again. The persistence of AAV vectors in animals has been studied.^{60,172}

The vector may optionally contain a DNA sequence encoding a mutant or modified β₂AR that is modified as compared to the native β₂AR or wild-type β₂AR. The modified β₂AR possesses at least one property that is different from the native β₂AR. For example, the modified β₂AR may possess increased responsiveness to β₂AR agonists, increased affinity to β₂AR agonists, and/or the capability to increase the potency of β₂AR agonists to simulate downstream signal transduction pathways, as compared to the native β₂AR. The modified β₂AR is modified from the native β₂AR by any one of or a combination of the following modifications, to include deletion of amino acids, substitution of amino acids, and/or replacement of amino acids. The modified β₂AR is produced by modifing the DNA sequence encoding the β₂AR prior to inserting the sequence into the vector.

For example, β₂AR mutants within the scope of the present invention are mutant β₂ARs with phosphorylation sites removed or a constitutively active mutant β₂AR.

A mutant β₂AR with phosphorylation sites removed is useful in the present invention. After binding hormone, the β₂AR rapidly loses its ability to respond to subsequently administered hormone. This process is commonly referred to as desensitization. Desensitization is mediated by G-protein-coupled receptor kinases (GRK) and arresting. Following hormone binding, the β₂AR is phosphorylated by a GRK which in turn leads to binding of arrestin. The binding of arrestin has two effects on β₂AR function, both of which diminish responsiveness to β₂-agonists. First, arrestin associated with the β₂AR prevents the β₂AR from interacting with the stimulatory guanine nucleotide regulatory protein G, and activating downstream signaling events. Second, arrestin binding to the β₂AR increases the likelihood that the receptor will be removed from the cell surface and internalized. This reduces the total number of β₂AR on the cell surface and reduces the responsiveness of the cell to β₂-agonist. Based on all of this information, the removal (or replacement) of the amino acids of the β₂AR that are phosphorylated by GRK prevents the process of desensitization. This concept is similar to related receptors for other hormones.¹⁷³ To prevent phosphorylation of the β₂AR by GRK, the receptor is either truncated (e.g., remove a portion of the carboxy tail that contains the phosphorylation sites or replace the serine and theronine residues in the carboxy tail with alanine and glycine residues. The amino acids serine and theronine are phosphorylation substrates. ^{173, 174}

Constitutively active mutant β₂ARs are also useful in the present invention. Normally, in order to observe activation of downstream signal transduction pathways (activation of adenylyl cyclase and cyclic AMP production) by the β₂AR, hormone must bind to the receptor. However, a report was published several years ago that reported a mutant β₂AR that activated adenylyl cyclase constitutively.¹⁷⁵ The mutant was created by replacing the carboxy terminal portion of the third intracellular loop of the β₂AR with the corresponding region of the α_{1B}-adrenergic receptor, a related receptor subtype that also binds epinephrine and norepinephrine. The resulting mutant receptor was expressed in COS-7 and CHO cells in vitro and tested for activity. The constitutively active β₂AR displayed the following characteristics: 1) an increased affinity for agonists, and 2) increased potency of agonists in stimulating adenylyl cyclase activity. Both effects are desirable in the present invention.

The vector of the present invention contains a promoter that is operably linked to the β₂AR gene and is functional in the cells to which the vector is administered. These cells include the airway epithelial cells and smooth muscle cells and the blood vessel endothelial cells and smooth muscle cells. Preferably, the promoter is a viral vector promoter or a mammalian cell specific promoter. If the promoter is a mammalian cell specific promoter, it is preferably is an epithelial cell specific promoter, an endothelial cell specific promoter, or a smooth muscle cell specific promoter. For example, a promoter was developed that directs expression of the human cystic fibrosis transmembrane conductance regulator gene to airway epithelial cells.²¹

The preferred promoter is a viral vector promoter, which is functional in mammalian cells including either epithelial, endothelial or smooth muscle cells. Examples of such viral promoters are a cytomegalovirus (CMV) promoter or an adeno-associated vector (AAV) promoter. More preferably the vector is an AAV vector containing a CMV promoter. The promoter is selected to obtain the maximum expression of the β₂AR gene in the transfected cells.

The promoter may alternatively be an inducible promoter that allows the regulation of the amount of receptor that is expressed. Such a promoter can be induced or upregulated by hormones or by other pharmacological agents. The inducible promoter preferably should be a weaker promoter, such as the endogenous β₂AR gene promoter or a tissue-specific promoter. The present invention is intended to encompass the administration of the inducer of the promoter to a human subject to induce the expression of β₂ARs in the target cells. The administration of the inducer in a pharmaceutical composition occurs at the same time that the vector comprising the DNA sequence encoding a β₂AR operably linked to the β₂AR endogenous promoter or a tissue-specific promoter so that the β₂AR gene is expressed. However, after the target cells are transduced and stably carrying the β₂AR gene, to obtain increased β₂AR expression in the target cells, it may be necessary to administer only the inducer or the inducer in combination with the β₂-agonist, to provide enhanced dilation of the airways or blood vessels of the subject.

Suzuki et al.¹³⁷ provide an example of a regulatable promoter that can be upregulated by exogenous agents that raise the intracellular levels of cAMP. In addition to a promoter, the vector optionally may contain at least one enhancer or regulatory element that allows the β₂AR gene to be turned on or off in the target cells. Burcin et al. ¹⁷⁶ discloses a regulator to a liver-specific promoter.

The application of gene therapy to treat human disease, while relatively simple in concept, is composed of series of challenges and problems that must be solved before beneficial effects can be realized. Two forms of gene therapy are currently used, ex vivo and in vivo. In ex vivo gene therapy that has been used in cancer patients¹⁴⁴, cells are removed from the patient, transfected with the transgene and then re-implanted into the patient. Gene therapy for tissues that cannot be easily removed from the patient and reimplanted requires using in vivo gene therapy.¹⁴⁶ Airway epithelium falls under the second category making in vivo gene therapy the required route of administration. Because all viruses transfer and express genetic material during their life cycles, they have frequently been used as vectors in in vivo therapeutic gene transfer protocols. Using treatment of cystic fibrosis patients with the cystic fibrosis transmembane conductance regulator (CFTR) gene as a model, several human gene therapy trials have shown that is possible to obtain expression of transferred genes to humans by a number of in vivo strategies²⁶. The three principle viral vectors used in cystic fibrosis therapy have been retroviruses, adenoviruses, and adeno-associated viruses. Each has its advantages and disadvantages.^{43,44} The present inventors have chosen an AAV vector because of 1) natural tropism towards airway epithelium, 2) efficient integration into the genome of nondividing cells, 3) lack of association with any known human disease, and 4) ability to express the transgene longterm without inactivation in vivo.

Recently, there has been increasing use of AAV in human gene therapy protocols. Currently, AAV is being tested in Phase I clinical trials with cystic fibrosis patients at the University of Florida and Johns Hopkins University. Initially, it appeared that there are no deleterious effects to overexpression of the CFTR protein in transgenic animals,¹³⁷ and therefore in early gene transfer approaches to cystic fibrosis the prevailing strategy was to express as much CFTR as possible. Therefore many transgenes were typically driven by strong viral promoters to obtain maximal expression levels.¹³⁷ More recently it has been recognized that the use of regulatable promoters may be a useful approach to increase safety and/or efficacy in human gene therapy protocols.^{51,61,137} Consequently, the present invention is intended to encompass the use of a regulatable promoter featuring a GRE to drive expression of the β₂AR transgene. The β₂AR gene is relatively small (^{˜}2 kb) and has no introns. The entire transcription cassette that is inserted in AAV, including the β₂AR gene and the neomyocin resistance gene, is approximately 3.6 kb, well under the packaging limit of 5.2 kb for efficient replication of AAV.⁶³

The present vector and methods also are useful in treating vascular diseases. Vascular tone of the pulmonary arteries is a consequence of pulmonary vasorelaxation and vasoconstriction.^{177,178} The pulmonary vasculature expresses both α-adrenergic receptors and β-adrenoreceptors, both of which help to regulate pulmonary vascular tone by producing vasoconstriction or vasodilation, respectively.¹⁷⁹ Pulmonary hypertension starts out with vasoconstriction of the small and medium size pulmonary arteries. As pulmonary hypertension progresses, there is vascular remodeling as a result of smooth muscle hypertrophy. At the level of the pulmonary arteries, there are only two cell types that have any known effect on arterial diameter: smooth muscle cells and endothelial cells. Vasorelaxation is achieved through pathways that are dependent or independent of the endothelial cells. The β₂-agonist, isoproterenol, produces vasorelaxation by interacting with β₂ARs on pulmonary vascular smooth muscle cells. Other agents, such as acetylcholine, produce vasorelaxation by stimulating endothelial cells to produce nitric oxide, which in turns, causes vascular smooth muscle cells to relax. The reason typical vasodilators such as β₂-agonists cannot be used to treat pulmonary hypertension is that they also cause arteries in the systemic circulation to dilate. This causes a precipitous drop in blood pressure and would lead to circulatory shock and death. The administration of β₂AR containing vector to the smooth muscle cells of the pulmonary arteries is the appropriate method, which results in increased β₂AR levels in these cells, making them more sensitive to circulating β₂-agonists. Low doses of β₂-agonists optionally are given, which cause pulmonary artery smooth muscle cells to relax, and thereby increase arterial diameter and reduce pulmonary arterial pressure. At this low dose of β₂-agonist, the dose is too low to appreciably affect the β₂ARs in the peripheral circulation detrimentally.

To target the vector containing the β₂AR gene to the pulmonary arterial smooth muscle cells, it is injected into a vein, for example the jugular vein. Venous blood returns to the right heart. This blood is then pumped to the lungs where it is oxygenated. Although the endothelial cells form a fairly tight barrier to the diffusion of anything including viruses, an appropriately designed gene delivery vehicle would transport the vector to the targeted cells.

The examples set forth below serve to further illustrate the present invention in its preferred embodiments, and are not intended to limit the present invention. The examples utilize specific vectors and nucleic acid sequences but it is intended that the vectors and nucleic acid sequences with similar functional properties are also intended to be encompassed by the present invention.

### EXPERIMENTAL EXAMPLES

### Construction of AAV Vectors

System for Producing Recombinant Adeno-Associated Virus

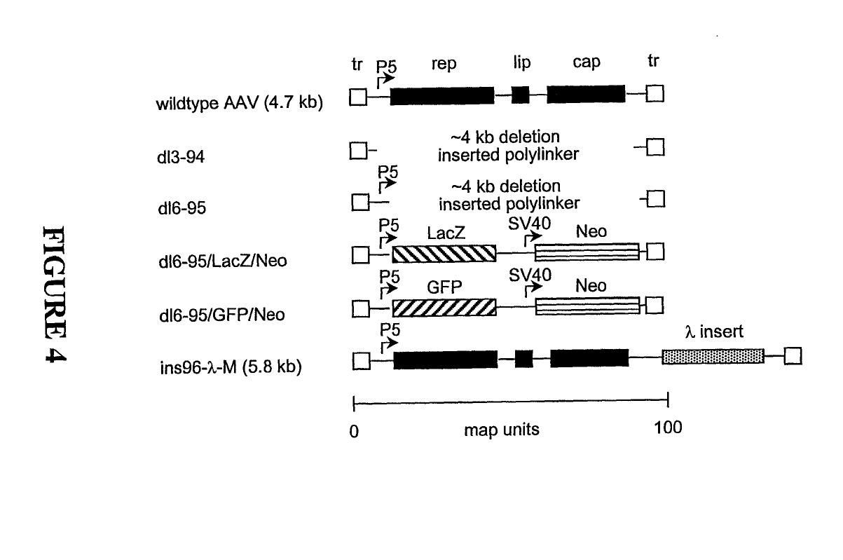

Viral vectors and their application to gene therapy are known. The original gene mapping and phenotype determinations in AAV,⁶⁴ were first published using recombinant AAV to transduce mammalian cells in vitro,⁶² and reported transduction of of hematopoietic stem cells with recombinant AAV,⁷⁶ and reported the maximum packaging capacity of AAV.⁶³ Several AAV genomes are useful as vectors to transfer genes into mammalian cells (FIG. 4). These include d13-94 which includes the terminal repeats, poly [A⁺] motif, and a unique BglII cloning site,⁶⁴ d16-95 which includes the same features as d13-94 plus the AAV P5 promoter⁶⁴ which allows constitutive expression of inserted transgenes. d16-95/LacZ/Neo allows color selection and d16-95/GFP/Neo allows expression of green fluorescent protein for detection of infected living cells. The AAV terminal repeats are the only cis sequences in the AAV genome that are required for DNA replication, packaging and integration into the host genome. Approximately 5 kb of DNA can be packaged into d16-95 and d13-94.⁶³ The Neo cassette can be removed and replaced with “filler DNA” for applications in which neomycin expression is not desirable (e.g., in vivo transduction of airway epithelial cells).

For generating recombinant AAV stock, a system was developed by Hermonat and Muzcyzka⁶² which uses a “wild-type”, replication competent AAV genome called ins96-λ-M (U.S. Pat. No. 5,139,941) (see FIG. 4 for structure). This AAV variant contains a 1.1 kb λ phage DNA insert at map unit 96 and consequently is too large to be packaged effectively, but promotes packaging of recombinant AAV. This system produces consistently high titers of recombinant AAV (^{˜}10⁵-10⁶ IU/ml compared to ^{˜}10³-10⁵ IU/ml) when a non-replicating complementor AAV is used.¹²² Using this method, wild-type AAV is produced at a level that is ^{˜}10-20% of that of recombinant AAV, an outcome that is not desirable in preparing recombinant AAV for human trials. However, in experiments presented herein, the presence of low levels of wild-type AAV will not affect the outcome of our experiments.

Improvements in vector titer and purity are necessary for use in human gene therapy applications. A method of recombinant adeno-associated virus production has been described that is completely free of helper adenovirus.¹⁶⁵ Use of this method to produce recombinant adeno-associated virus is advantageous in that a more defined reagent will result that is less likely to produce a host immune response. Further improvements in the methodology for recombinant adeno-associated virus preparation are likely as use of the vector in human gene therapy increases.

It is recognized that there are alternative ways to construct recombinant AAV vectors but for maximal expression of the β₂AR, a viral promoter is preferred, such as the CMV promoter or the AAV P5 promoter; or for inducible expression, the endogenous β₂AR promoter, together with the composite GRE identified in this application is selected.

The AAV P5 promoter is small and is contained as a convenient cassette with the AAV replication origin and the AAV terminal repeats (TR) which must be included in any AAV-transducing vector.⁹⁴ Many other constitutive promoters (e.g., SV40, RSV-LTR) also are useful in the present invention. The β₂AR transcription cassette can be modifed by altering the sequence, and the number of GREs as well as adding other transcriptional elements to improve inducibility. The β₂AR promoter may be modified to include an epithelium-specific expression cassette.²¹ This cassette includes regulatory elements from the human cytokeratin gene. It has been used to efficiently express reporter genes as well as the human cystic fibrosis transmembrane conductance regulatory protein (CFTR) in airway epithelial cells.²¹ Other useful promoters that drive β₂AR expression are the human surfactant protein C promoter or the CC10 promoter. These promoters have been used to drive β₂AR gene expression in the airways of transgenic mice.⁸⁹ Any human promoter effective in the rat-derived SPOC1 cell line is useful in the present invention. Because AAV displays tropism for airway epithelium, an epithelial cell-dependent promoter is not necessary in order to achieve expression of the β₂AR transgene in airway epithelial cells. In fact, adeno-associated viruses have been used to transfer the CFTR gene into airway epithelial cells.^{43,44,45} SPOC1 cells are derived from airway epithelium and are readily infected by AAV. However, alternative methods to transfer DNA into cells also are used. These include using adenovirus, used to transfer the CFTR gene to airway epithelium,⁵⁴ guanidinium-cholesterol cationic lipids¹⁰⁸ or by targeting the polymeric immunoglobulin receptor.⁴¹ Finally, radioligand assays and cyclic AMP radioimmunoassays are routine procedures to assess the functional outcome of β₂AR overexpression in SPOC1 cells.

Specific Methods

AAV Constructs containing Rat β₂AR Gene

To study transcriptional regulation of β₂AR gene expression, a rat β₂AR genomic clone was inserted into the EcoRI site of λ phage Charon 4A from Dr. P. Buckland, University of Wales. The clone includes the 4190 bp section submitted to the Genebank/EMBL database,¹⁶ plus an additional unsequenced section of aproximately 1400 bp in the 5′-flanking region.

Cell Culture: Tracheal Epithelial Cell Line SPOC1 and 293 Cells

The rat tracheal epithelial cell line, SPOC1, are used for experiments described in this section and are maintained in cell culture as described herein. The human kidney carcinoma derived 293 cell line is maintained in Iscove's modified Dulbecco's media supplemented with 10% fetal bovine serum. Preliminary experiments are conducted in order to establish the optimal conditions for infection of SPOC1 cells with AAV.

Generation of SPOC1 Cells Expressing Green Fluorescent Protein

Optimal conditions for infection of SPOC1 cells with AAV are established using a recombinant AAV that expresses green fluorescent protein. This construct, d16-95GFP, expresses green fluorescent protein under the control of the viral P5 promoter.¹⁵⁸ SPOC1 cells are infected with d16-95GFP at a multiplicity of infection (MOI) of at least 1. Forty-eight hours post infection, cells are observed using a Zeiss Axiovert inverted microscope equipped for epifluorescence illumination with Hammamatsu chilled CCD and Contax 35 mm cameras for image collection in order to determine the transduction frequency. For time-lapse imaging of living cells, the microscope is also fitted with Bioptechs culture dish and objective temperature controllers. Digital image acquisition is controlled with Cell Robotics Workstation software. To prevent photobleaching, a filter wheel is programmed to block the excitation illumination between exposures.

Expression of Green Fluorescent Protein (GFP) in SPOC1 Cells Transduced with Recombinant AAV Carrying the Green Fluorescent Protein (GFP) Gene

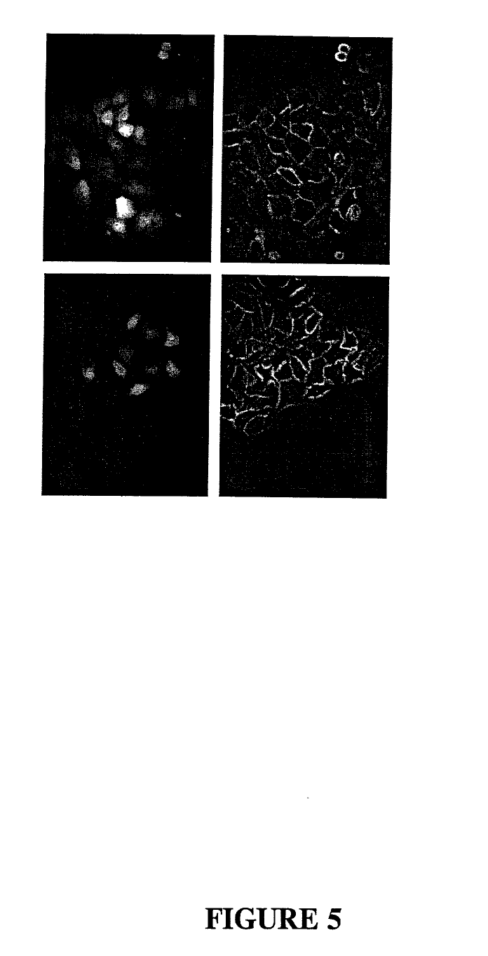

SPOC1 cells were infected with d16-95GFP/Neo which expresses green fluorescent protein under the control of the viral P5 promoter (FIG. 4). Seventy-two hours post infection, cells were observed using a Zeiss Axiovert inverted microscope equipped for epifluorescence illumination. Cells were observed using both interference contrast and epifluorescent illumination. As shown in FIG. 5, transduction of SPOC1 cells by recombinant AAV was relatively efficient. Panels A and B show fluorescent images obtained from two different groups of cells. GFP fluorescence was observed in a mostly diffuse pattern throughout the cytoplasm (FIG. 5). The same cells visualized by interference contrast microscopy are shown in Panels C and D (FIG. 5). Overall, greater than 50% of the cells were transduced by the recombinant AAV as judged by GFP fluorescence. These results demonstrate that the SPOC1 cell line can be transduced by AAV.

Preparation of Recombinant AAV Containing a β₂AR cDNA

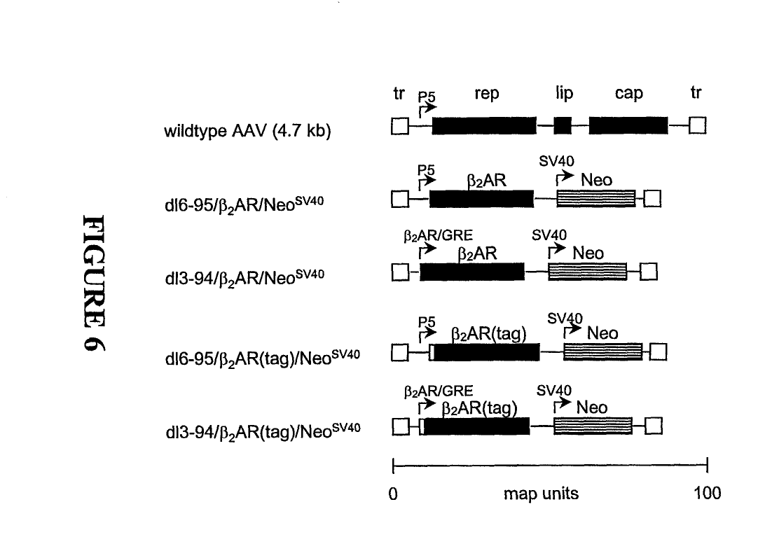

Four different recombinant AAV vectors (for structures see FIG. 6) are prepared. As an example, only the construction of one recombinant AAV vector, d16-95/β₂AR/Neo^{SV40}, is described here. The starting point is pd16-95PL1 which contains the AAV terminal repeats, the AAV P5 promoter, and a polylinker with multiple cloning sites with two interspersed poly [A⁺] signals. Into this plasmid is inserted the Neo gene to form the parental plasmid, pd16-95/Neo, which contains an SV40 EPR-NeoR transcription cassette on a 1.2 kb XbaI fragment ligated into the XbaI site of pd16-95PL1 (both pd16-95PL1 and pd16-95/Neo obtained from Dr. Hermonat). The NdeI end of a 1.7 kb HindIII/NdeI fragment encoding the rat β₂AR is converted into a HindIII site with a linker for cloning into the HindIII site of pd16-95/Neo. Recombinants are sequenced in order to identify clones with the β₂AR coding sequence is in the proper orientation. The combined size of Neo and the β₂AR sequence, approximately 3.6 kb, is well under the maximum insert size for efficient AAV replication.⁶³ The construct is transformed into competent E. coli and positive clones selected and purified using a plasmid mini-prep kit (Promega, Madison, Wis.).

The four types of recombinant AAV vectors are prepared, all of which will include the coding region of the β₂AR (see FIG. 6 for structures): 1) d16-95/β₂AR/Neo^{SV40}, 2) d13-94/β₂AR/Neo^{SV40}, 3) d16-95/β₂AR(tag)/Neo^{SV40}, and 4) d13-94/β₂AR(tag)/Neo^{SV40}. Constructs d16-95/β₂AR/Neo^{SV40} and d16-95/β₂AR(tag)/Neo^{SV40} include the rat β₂AR cDNA whose expression are under the direction of the AAV P5 promoter which allows high-level, constitutive expression of the β₂AR in infected SPOC1 cells. Constructs d13-94/β₂AR/Neo^{SV40} and d13-94/β₂AR(tag)/Neo^{SV40} include the β₂AR cDNA whose expression is under the direction of the β₂AR promoter and the composite GRE previously identified. Expression of these transgenes enable regulation of expression by glucocorticoids. Two constructs carry the β₂AR cDNA with an epitope tag attached to the carboxy terminus to allow recombinant β₂AR to be distinguished from native β₂AR. All AAV vectors will carry the Neo gene under the control of the SV40 early promoter to allow selection of AAV-infected SPOC1 cell colonies under the antibiotic G418. SPOC1 cells are transduced with these vectors, then assays (radioligand assays, cyclic AMP determinations) are performed to assess levels and function of expressed β₂ARs. Radioligand assays are performed to establish the total number of β₂ARs (native and recombinant) on SPOC1 cell surface. Levels of recombinant β₂AR are determined by antibody detection of the epitope tag. Functional coupling of SPOC1 cell β₂ARs to downstream signal transduction pathways are assessed by a cyclic AMP radioimmunoassay.

The inducibility of d13-94/β₂AR/Neo^{SV40} and d13-94/β₂AR(tag)/Neo^{SV40} are tested by incubating SPOC1 cells infected with these vectors with the synthetic glucocorticoid dexamethasone. Radioligand assays and antibody detection of the epitope-tagged β₂AR are performed to determine β₂AR levels in transduced cells. Cyclic AMP radioimmunoassays are performed to determine functional coupling of expressed β₂ARs.

Packaging and Titering of Recombinant AAV Virus Stocks

Recombinant virus stocks are generated using the AAV complementor genome ins96-λ-M as previously described.⁶² This complementor genome has all the AAV genes and regulatory sequences necessary for replication, but has a 1.1 kb λ phage insert located in a non-essential site at map unit 96. Because of the presence of the insert, the ins96-λ-M genome is inefficiently packaged into virions, but recombinant AAV is packaged. Low levels of wild-type AAV are produced by this procedure, an inconsequential outcome since AAV is non-pathogenic. Detailed methodology can be found in Hermonat and Mazyczka.⁶² Briefly, recombinant vector plasmid (5 μg) are DEAE/Dextran transfected along with ins96-λ-M plasmid into 293 cells. Various cell lines can be used for packaging, but 293 cells display a high efficiency of transfection.² Cells are then infected with AAV type 2 at a MOI of 5. Forty-eight hours later, at maximum cytopathic effect, the cells are lysed by freeze-thawing the plates three times, followed by heating to 56° C. to kill the virus. After a low-speed centrifugation to remove cellular debris, a homogeneous recombinant vector preparation free of wild type AAV is obtained by multiple CsCl₂ equilibrium gradient centrifugations. Recombinant virus stocks are titered by performing Southern blot hybridization of isolated single-stranded vector DNA to determine copy number of packaged genomes¹²² and by comparing the generation of G418 resistant colonies of the untitered virus stock compared to that of known titers of wild type AAV virus stock.⁶²

Infection and Selection of SPOC1 Cells

The recombinant AAV viruses are used to infect SPOC1 cells at a MOI of either 1 or 10. In addition, SPOC1 cell cultures are mock-infected. Cells are exposed to virus for 1 hr at 37° C. and then plated in 2 ml of media for continued culture at 37° C. G418 (final concentration=400 μg/ml) is added to the medium 48 hr after infection. After three weeks of G418 selection, neomycin resistant cells are cloned and expanded. To determine if stable integration of the β₂AR gene is present in G418 selected SPOC1 cells, DNA is extracted, digested with HindIII and BglII and Southern blot analysis is performed using standard methods.⁸⁶ Detection of a −1.7 kb fragment that hybridizes with a radiolabeled β₂AR cDNA would indicate stable integration of the recombinant β₂AR gene.

Epitope-Tagged β₂-Adrenerzic Receptor

Because SPOC1 cells express a wild-type β₂AR, it is useful to have a method to detect the expression of recombinant β₂AR in clonal lines infected with recombinant AAV. To accomplish this, an epitope-tagged β₂AR is used. The cDNA encoding the rat β₂AR are modified by insertion of the sequence encoding YPYDVPDYA at the amino terminus of the receptor by oligonucleotide-directed mutagenesis. This modification has been performed on the human β₂AR and has been shown to not alter expression or function of the receptor.¹⁴⁷ This nine amino-acid epitope is recognized by the antibody 12CA5.¹⁰⁰ Thus, immunoblot analysis of membrane fractions prepared from SPOC1 cells can be used to detect recombinant receptor. Membrane fractions from infected SPOC1 cells are resolved on 10% SDS polyacrylamide gels, transferred to nitrocellulose filters (Schleicher and Schuell, Keene, N.H.). Immunoblotting is performed in 5% nonfat dry milk containing 2% Nonidet P-40 as previously described using primary antiserum at 1/600 and horseradish peroxidase-conjugated second antibody. ¹⁴⁷ The presence of recombinant β₂AR in clonal cell lines infected with recombinant AAV vector was detected, whereas mock-infected cells did not express the epitope-tagged β₂AR.

Treatment of SPOC1 Cells with Dexamethasone

Two of the β₂AR transgenes that are under the control of the β₂AR gene promoter plus the composite GRE is inducible by dexamethasone. Clonal SPOC1 cells that had been infected with either the AAV vector d13-94/β₂AR/Neo^{SV40} or d13-94/β₂AR(tag)/Neo^{SV40} and mock infected cells are treated with either vehicle or 0.5 μM dexamethasone for 12 hours. Membrane fractions are prepared in order to determine β₂AR number. In separate experiments, cells subjected to the same treatments are treated with (−)-isoproterenol and cyclic AMP concentrations determined by radioimmunoassay. In experiments with SPOC1 cells infected with d13-94/β₂AR(tag)/Neo^{SV40}, levels of the epitope-tagged β₂AR are determined by immunoblots. β₂AR levels and β₂AR-stimulated adenylyl cyclase activity is increased in cells treated with dexamethasone. These experiments are repeated six times with six different cell platings to perform statistical analysis of the results.

β₂-Adrenergic Receptor Radioligand Assays

In order to determine levels of β₂AR expression in clonal SPOC1 cells, radioligand assays with [¹²⁵I] cyanoiodopindolol ([¹²⁵I]CYP) are performed. Partial purified membrane preparations are obtained from mock-infected and clonal SPOC1 cells by differential centrifugation essentially as previously described.¹⁰⁴ Briefly, cells are washed with ice-cold phosphate buffered saline (PBS) and scraped into ice-cold PBS with a rubber policeman. The cells are centrifuged at 250×g for 5 min, resuspended in assay buffer (50 mM Tris HCl, pH 7.4, 2 mM MgCl₂), and homogenized with a glass-glass homogenizer followed by sonication (five 10 second bursts at setting 6) with a Tekmar Model AS1 Sonic Disrupter. The nuclei are removed by centrifugation at 600×g for 10 min. The membranes are obtained from the resulting supernatant by centrifugation at 30,000×g for 15 min. The membranes are resuspended in assay buffer and centrifuged again at 30,000 g for 15 min. The final pellets are resuspended in assay buffer, aliquoted and stored at −80° C. until used for radioligand assays. Protein concentrations of membrane preparations are determined by the method of Bradford¹¹ using bovine serum albumin as the standard. Typically, ^{˜}70 μg of membrane protein from SPOC1 cells is obtained that are at 80% confluency in 100 mm dishes. Thus, six plates yield sufficient membrane protein in order to perform either one saturation experiment or a single competition curve. [¹²⁵I]CYP (Dupont-NEN, Boston, Mass.; specific activity=2200 Ci/mmole) are used to identify β₂ARs as previously described.⁸⁷ In saturation experiments, aliquots of SPOC1 cell membranes (final protein concentration in the assays tube=16 μg/ml) are incubated with eight different concentrations of [¹²⁵I]CYP ranging from 0.5 to 80 pM. Nonspecific binding is defined with 0.1 μM (−)-propranolol. Data from saturation experiments are analyzed using the LIGAND program to obtain binding site concentration and the dissociation constant for [¹²⁵I]CYP. Since [¹²⁵I]CYP cannot distinguish between native and recombinant β₂AR, observed increments in total β₂AR levels in clonal cells compared to that in mock-infected SPOC1 cells are attributed to arising from the virally-introduced recombinant β₂AR gene. In order to characterize pharmacologically the expressed recombinant β₂AR in clonal SPOC1 cells, competition experiments with β-adrenergic agonists and antagonists are performed. In competition experiments, aliquots of SPOC1 cell membranes (final protein concentration in the assay tube=16 μg/ml) are incubated with a single concentration of [¹²⁵I]CYP (^{˜}1 pM) and increasing concentrations of the competitor. Inhibition constants for agonists and antagonists are calculated using the method of Cheng and Prusoff²⁰ and are compared with that of wild-type β₂AR. Saturation and competition experiments with each agonist and antagonist are performed six times with different membrane preparations in order to obtain accurate estimates of binding site concentration and inhibition constants for agonists and antagonists. The results of these experiments show the [¹²⁵I]CYP binding site in clonal SPOC1 cells as the β₂AR subtype.

Cyclic AMP Radioimmunoassay

In order to determine if the recombinant β₂AR expressed in clonal SPOC1 cells is able to functionally couple to adenylyl cyclase, cyclic AMP radioimmunoassays are performed using lysates. Mock-infected and clonal SPOC1 cells are plated at a density of 100,000 cells/well in 12-well dishes (Costar, Cambridge, Mass.). Cells are treated with adrenergic agonists and the phosphodiesterase inhibitor isobutylmethylxanthine (IBMX) for 10 min. Cellular cyclic AMP levels are determined by radioimmunoassay using the Biotrak CAMP Assay System (Amersham Life Science, Arlington Heights, Ill.). Agonist-stimulated cyclic AMP accumulation will indicate that surface β₂ARs are functionally coupled to adenylyl cyclase. Clonal SPOC1 cells overexpressing β₂AR would be expected to display increased sensitivity to β-agonist stimulated cyclic AMP formation. These experiments are repeated four times with four different SPOC1 cell platings.

AAV Constructs Containing the Human β₂AR Gene

Construction of an AAV-Human β₂AR Vector

Polymerase chain reaction (PCR) was performed on human genomic DNA known in the prior art^{73,171} to obtain the β₂AR gene, using a forward primer engineered with a HindIII restriction endonuclease cut site 5′ of the ATG, and a reverse primer engineered with a BamHI restriction endonuclease cut site 3-prime to the stop codon. The PCR product was phenol chloroform extracted twice and chloroform extracted once. Phases were separated by centrifugation in a Phase Lock I Light (5 prime 3 Prime, Inc. cat p1-175850), ethanol precipitated and resuspended in RE buffer E and cut with BamHI and HindIII (Promega Corp) for 2 hours at 37° C. The RE digest was cleaned up using the Wizard DNA clean-up Kit (Promega Corp., cat # A7280). The vector used as the recipient of the β₂AR gene PCR product was pCEP4 (Invitrogen, cat # V044-50). This vector provided the CMV promoter and the SV40pA tail. The insert was directionally cloned into the polylinker region via sticky-end ligation using the 2×rapid ligation buffer and T4 Ligase from the pGem-T Easy Vector System I using protocol instructions from that kit (Promega Corp., cat # A1360).

The CMV promoter—human β₂AR gene—SV40 pA tail moiety was released by digestion with SalI restriction endonuclease in D buffer (Promega Corp.) for 2 hours in a 37° C. water bath. The digest was run on a 1% NuSieve GTG agarose mini gel (FMC BioProducts, cat # 50081) in lxTBE buffer at 100 volts for one hour and subsequently stained with ethiduim bromide to visualize the DNA bands. The fragment of choice was excised under minimum UV exposure with a sterile razor blade and the agarose strip was placed in a 1.5 ml microfuge tube and melted in a 65° C. water bath for 30 min. The DNA fragment was isolated from the melted agarose using the Wizard PCR Clean-up kit (Promega Corp., cat # A7170).

The AAV vector used to accept the CMV promoter—human β₂AR gene—SV40 polyA tail moiety was pAV53-LR, the cloning vector containing the ITR's from AAV (obtained from Jianyun Dong, University of South Carolina). This vector was linearized with XhoI restriction endonuclease in buffer D (Promega, Corp.) for 2 hours in a 37° C. water bath. The digest was cleaned up using the Wizard DNA Clean-up kit (Promega Corp. cat # A7280).

The gel-purified Sall fragment insert from the pCEP4/human β₂ gene and the linearized pAV53 LR vector were ligated using sticky end ligation protocol from the T Easy Vector System I, (Promega Corp. cat # A1360) overnight at 4° C. (XhoI and Sall have compatible ends.)

The size of the insert in pAV53-LR needs to be between 4.0-4.8 kb. Up to a 1.9 kb fragment was needed to achieve an insert within the optimal size range. The pEGFP-C1 cloning vector (Clontech Laboratories, Inc., cat # 6084-1) provided sufficient base pairs of the marker gene with it's own promoter and polyA tail to use as a DNA filler insert for the pAV53-LR/CMV-Huβ2AR-SV40pA to obtain an optimal cassette length. The CMV IE promoter, EGFP gene—SV40 poly A tail was PCR-amplified out of the pEGFP-C1 vector using primers (Biosynthesis) engineered with SphI sites in the forward and reverse directions. The PCR product was phenol chloroform extracted and ethanol-precipitated as previously described and resuspended in deionized, double distilled water and subsequently digested in a 37° C. water bath with SphI restriction endonuclease in buffer K (Promega Corp.) and cleaned up using the Wizard DNA clean-up kit (Promega Corp., cat # A7280).

The EGFP insert was ligated to the pAV53 LR/CMV-Huβ₂AR-SV40pA vector, pre-linearized with SphI restriction endonuclease, by sticky end ligation as previously described. This final vector has a 2,610 bases inserted between the ITR's of the pAV53-LR vector. The total DNA cassette length is 4651 base pairs and codes for the human β₂AR and the Enhanced Green Fluorescent Protein.

Adenovirus production

HeLa cells (ATCC cat # CCL-2) are grown in in two 10 ml dishes in DMEM medium (CellGro cat # 10-013-CM) with 10%FBS (Gibco cat # 16000-044) added, and incubated in a 37° C./5% CO₂ (Forma Scientific water jacketed tissue culture incubator).

Adenovirus stock (AV) obtained from Jianyun Dong, PhD's laboratory at the University of South Carolina was added to infect both plates of 90-95% confluent HeLa cells grown overnight −(25 μl/10 cm dish, and then incubated at 37° C. 5% CO₂ in a tissue culture incubator for 24 hours. These plates are examined closely over the next 12 hours to determine cytopathic effect (CPE). At approximately 50% CPE, all the media is removed from the plates and washed one time with 10 ml, serum-free DMEM. Then 1 ml of serum free DMEM is added and the cells are harvested to a 1.5 ml eppendorf microfuge tube by gently scraping with rubber policeman. The cells are frozen in liquid N₂ for 2 min and then thawed in 37° C. water bath for 3 min. This cycle is repeated for a total of 3 freeze/thaw cycles to crack apart the cell membranes. The cell membrane debris is pelleted for 5 min and aliquots of the supernatant are collected in 50 μl aliquots and stored at −80° C.

To determine titer of AV harvested above, HeLa cells were grown overnight in 12-well dish at about 80% confluent, in 0.5 ml DMEM/10%FBS medium/well. The next morning, one tube of AV stock was thawed and dilutions made (1:10 and 1:100 in serum-free DMEM). Increasing amounts of diluted AV (2-8 μl of 1:10 dilution, and 2-50 μof 1:100 dilution) were added to each of the 12 wells. The cells were placed in the 37° C./5% CO₂ tissue culture incubator. At 48 hours, the cells were observed, and the wells showing 50% CPE were the optimal amount of AV to use for transfection of the AAV constructs into HeLa cells for the packaging of the AAV.

Transduction of HeLa Cells with the AAV Constructs

HeLa cells were seeded in 6-well plates at approximately 50-80% confluency and grown at 37° C./5% CO₂ overnight. The cells were transfected using the LipofectAMINE plus protocol (Gibco cat # 10964-013) and Optimem transfection medium (Gibco cat # 31985-062). The ratio of helper to AAV vectors was 10:1 as previously determined by Jianyun Dong's laboratory. At the end of the DNA:lipofectamine complexing incubation period, the predetermined amount of diluted AV stock was added to the lipofectamine:DNA complex immediately prior to putting on the cells. The plates of transfecting cells were placed in the tissue culture incubator (37° C./5% CO₂) for 4 hours. An equal volume of DMEM medium containing 20% FBS was added to each well for a final concentration of 10% FBS, and then incubated for up to 48 hours at 37° C./5% CO₂. The media was removed and the plates tapped to dislodge cells. The cells were pooled to a 1.5 ml microfuge tube (Sarstedt cat # 72.690) in a total volume of 1 ml serum-free media, and then frozen in liquid N₂ for 3 min, thawed in a 37° C. water bath for 3 min, vortexed. The freeze/thaw/vortex cycle was repeated for a total of 3 times. Cellular debris was pelleted in a tabletop microcentrifuge (Eppendorf 5415-C) at room temp for 5 min . The supernatant was transferred to a new 1.5 ml microfuge tube and then stored (4° C. for up to one week, or −80° C. if to be used later than one week). Note—before freezing: The AV is inactivated by heating the above virus harvest at 56° C. for 60 minutes.

The above harvested virus was added directly to overnight growths of SPOC1 cells that were seeded at approximately 85% confluency in defmed medium as below:

Base medium is F-12/DMEM (Gibco cat #11320-033)

5 μg/ml Insulin (Sigma cat # I 6634)

0.1 μg/ml Hydrocortisone (Sigma cat # H 0135)

5 μg/rml Transferrin (UBI cat # 04-101)

5 ng/ml EGF (UBI cat # 01-107)

0.1 μg/ml Cholera Toxin (Sigma cat # C 3012)

50 μM Ethanolamine (Sigma cat # E 6133)

50 μM Phosphoethanolamine (Sigma cat # P 0503)

15 mM Hepes (Gibco cat # 11344-025)

1.5 mg/ml BSA (Sigma cat # A 2934)

30 μg/ml Bovine Pituitary Extract (UBI cat # 02-103) filtered through a 0.45 μm CA filter unit (Nalgene cat # 155-0045)

The Hela cells were grown overnight at 37° C./5% CO₂ and then viewed under a Fluorescent microscope at 24 and 48 hours to determine infection efficiency (MOI) using the marker gene, EGFP which is a part of the AAVβ₂Hu construct.

For the treatment of human subjects, it is important to remove the adenovirus helper prior to administration. The adenovirus can be removed using the methods of U.S. Pat. No. 5,139,941, and the new methods of U.S. Pat. Nos. 5,945,335; 6004,797 and 6,001,650. The present method is intended to utilize any method or to remove the adenovirus from the AAV-β₂AR stock. Additionally, the described AAV-β₂AR construct is useful to transduce human airway epithelial and smooth muscle cells but contains an inactivated phosphorus fluorescent green protein gene promoter. This protein gene is left in the construct to provide the preferred size of approximately 4.7 kb between the ITRs of the AAV vector.

Effect of Airway Epithelial Cell Directed β₂AR Gene Therapy in a Rat Model that Displays Airway Hyper-Responsiveness Following Antigen Challenge

The AAV vectors containing the β₂AR gene described above are used to transduce the epithelial cells of the airways of subjects, including rodents and humans, and the airway responsive is measured.

The studies described herein determine the effect of overexpression of the β₂AR in airway epithelial cells and its beneficial effect on airflow. The Brown-Norway rat was chosen as the experimental model because this inbred strain displays airway hypersensitivity to cholinergic agonists following sensitization and subsequent challenge with ovalbumin. The sensitized Brown-Norway rat is considered a reasonable approximation of the state of airways in atopic asthma.³⁷ A consideration is that the rats may develop an immunogenic response to the Neo gene product. Since this may result in additional airway inflammation (beyond that caused by the sensitization protocol) and expression of the Neo gene product is not needed in these in vivo experiments, the Neo cassette is inactivated by mutating the AUG that encodes the initiator methionine. This will disrupt the open reading frame. Alternatively, the Neo gene cassette is removed and replaced with spacer sequence in order to keep the size of the vector at −4.5 kb to optimize packaging efficiency. For purposes of clarity, the same nomenclature to describe the various AAV constructs that are used despite inactivation of the Neo gene cassette. Experiments are conducted to determine expression levels of the β₂AR transcription cassetteare retained. These experiments are performed to confirm that recombinant AAV vectors containing β₂AR transcription cassettes act by increasing recombinant β₂AR protein levels directly rather than by activating expression of the endogenous β₂AR gene. Modifications in the β₂AR transcription cassette optionally may be made to improve expression of recombinant β₂AR in airway epithelial cells.

Four separate studies are conducted to assess the physiological consequences of β₂AR overexpression in airway epithelial cells. The results of these studies determine the effect of β₂AR overexpression on lung function, the duration of the beneficial effect, the extent of recombinant β₂AR induction that can be achieved by administration of dexamethasone, and effect of withdrawal of glucocorticoids on β₂AR expression and lung function.

Specific Methods

Increased Airway Sensitivity to a Methacholine Challenge in the Ovalbumin-Sensitized Brown-Norway Rat

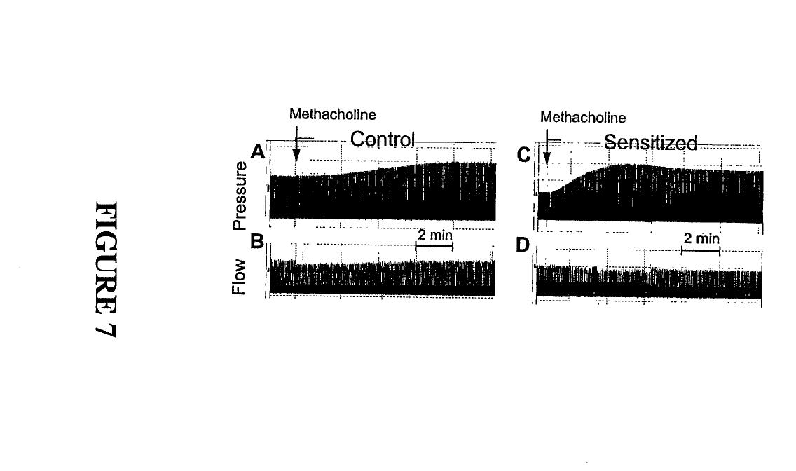

Experiments with control and ovalbumin-sensitized Brown-Norway rats are performed in which the airway sensitivity to a methacholine challenge was determined. Animals are sensitized and challenged. Inbred Brown-Norway rats (8-9 weeks old) are actively sensitized using ovalbumin by a standardized procedure. ¹¹⁷ Briefly, animals are sensitized by a single subcutaneous injection of 1 ml of 0.9% NaCl containing 1 mg ovalbumin and 200 mg aluminum hydroxide. Fourteen days later, animals are challenged with an aerosol of 1% ovalbumin delivered by a Marquest hand-held updraft nebulizer. Rats exposed to a single ovalbumin challenge following sensitization display a significantly increased responsiveness (an increase in measured airway resistance) to inhaled acetylcholine compared with saline-exposed rats.³⁸ This model of airway hyper-responsiveness has been established as described in the present invention (see, FIG. 7).

The protocol used to sensitize and then challenge Brown-Norway rats is the following:³⁸ Animals were injected with either 0.9% NaCl (control) or 1 mg ovalbumin and 200 mg aluminum hydroxide in 0.9% NaCl (sensitized). Two weeks later, control animals and ovalburnin-sensitized animals were exposed to aerosolized 0.9% NaCl or 1 mg/ml ovalbumin, respectively, for 30 min. The animals were anesthetized and instrumented exactly as described herein. Data from one control and one ovalbuminsensitized animal are shown in FIG. 7 Animals were placed on ventilators so that air flow is constant (FIGS. 7B and 7D, bottom panels). With the addition of methacholine, 1 mg/ml in nebulized form (FIGS. 7A and 7B) airway pressure increases. From the relationship Q=P/R, since flow (Q) is constant the increase in airway pressure must be due to an increase in airway resistance. Examination of the pressure traces, clearly demonstrate increased sensitivity of ovalbumin-sensitized Brown-Norway rats (FIG. 7C) to a methacholine challenge compared to the control rats (FIG. 7A). These results, consistent with previously published values for methacholine-induced increases in airway resistance in ovalbumin-sensitized Brown-Norway rats,¹¹⁷ demonstrate a reprodible model. From this data, reversal of methacholine-induced increases in airway resistance are easily measurable in ovalbumin-sensitized Brown-Norway rats that over-express the β₂AR following transduction with recombinant AAV.

Data Analysis

Representative tracings of air flow and pressure from control and ovalbumin-sensitized Brown-Norway rats that were subsequently challenged with aerosolized methacholine are shown in FIG. 7. Lung resistance is determined essentially as described³⁴ after subtracting the resistance of the endotracheal tube. The methacholine concentration given is that required to increase lung resistance to 200% of that measured in vehicle treated animals. Sensitivity to the airway relaxing effect of the β-adrenergic agonist (−)-isoproterenol are determined by administering increasing concentrations of (−)isoproterenol in a cumulative fashion. Airway resistance are calculated after each dose of (−)-isoproterenol which will allow a dose-response curve to be plotted and an ED₅₀ calculated for each experimental animal. Differences between the mean isoproterenol ED₅₀ of different experimental groups are compared by ANOVA followed by Newman-Keuls test. The accepted level of significance is 0.05.

Infection of Brown-Norway Rats with recombinant AAV Vectors

Administration of Recombinant AAV

Approximately 10¹⁰ AAV particles in 300 μl PBS are instilled intratracheally into anesthetized Brown-Norway rats as previously described. ¹⁵³ This procedure has been used successfully to achieve high level expression of transforming growth factor-β1 in bronchoalveolar fluids from rats that had been subjected to adenovirus-mediated gene transfer.¹³¹ Using similar methodology, airway epithelial cells have been transduced by AAV vectors in the rabbit.⁶⁰

Demonstration of AAV Infection Sites by In Situ Staining of LacZ

Because Brown-Norway rats have not been extensively used for studies on AAV-mediated gene transfer in the lung, preliminary experiments are performed in which d16-95/LacZ/Neo^{SV40} instilled intratracheally and 24 hours later the distribution and cellular localization of LacZ determined. A previously described method for histochemical staining for LacZ protein are used,⁸⁵ as modified by Xing et al.¹⁵³ Briefly, 24 hours after infection with d16-95/LacZ/Neo^{SV40}, animals are anesthetized, and lungs fixed by intratracheal perfusion with 2% formaldehyde containing 0.2% glutaraldehyde in PBS at 4° C. The lungs are rinsed twice by intratracheal perfusion with PBS and stained by intratracheal infusion of a solution containing 5 mM K₄Fe(CN)₆, 5 mM K₃Fe₃, 2 mM MgCl₂, and 0.5 mg/ml of the X-gal stain (5-bromo-4-chloro-3-indolyl-β-D-galactopyranoside) at 37° C. overnight. The stained lung tissues are then embedded in paraffm, sectioned, counterstained with nuclear red fast, and examined under the microscope for product formation. The results of these experiments will determine the major cellular sites of infection by AAV, likely epithelial cells of the small respiratory bronchioles.

Preliminary experiments are performed in which four Brown-Norway rats are infected intrtracheally with a recombinant AAV vector that contains a LacZ transcription cassette (d16-95/LacZ/Neo^{SV40}). Twenty-four to forty-eight hours later, animals are sacrificed and their lungs sectioned and treated with the substrate 5-bromo-4-chloro-3-indolyl-β-D-galactopyranoside. Sections are observed under the microscope and cells containing blue stain indicate the presence of LacZ. The results from these experiments allow the identification of recombinant AAV infection and the location of infected cells.

Localization of Recombinant AAV Transcription Cassette mRNA by In Situ Hybridization

In order to confirm that d13-94/β₂AR/Neo^{SV40} vector DNA acts by increasing recombinant β₂AR protein levels directly rather than by activating expression of the endogeneous β₂AR gene, in situ hybridization is used to determine expression levels of the β₂AR transcription cassette. In addition to β₂AR coding sequence, the recombinant mRNA will have unique sequences (e.g., polylinker and some viral sequence) to which anti-sense oligonucleotides are synthesized. The in situ hybridization is performed using anti-sense oligonucleotides as probes^{18,69} and these protocols are used to localize β₂AR transcription cassette mRNA in the lungs of recombinant AAV infected Brown-Norway rats.

Alternative experimental approaches than those described are also within the scope of the present invention. In place of, or in addition to, the experiments in which in situ hybridization are used to determine expression levels of the β₂AR transcription cassette, animals are infected with the inducible recombinant AAV vector d13-94/β₂AR(tag)/Neo^{SV40} or the consititutively expressed d16-95/β₂AR(tag)/Neo^{SV40}. Expression of the transgene is monitored by immunohistochemistry using a commercially available antibody directed against the tagged epitope as the first antibody. This approach allows expression of the transgene to be localized at the cellular level as well and provides direct evidence that MRNA transcribed from the β₂AR transcription cassette was indeed being translated into protein. Finally, the determination of airway resistance is the most appropriate physiological parameter to measure as a determinant of the beneficial effect of overexpression of β₂ARs in airway epithelial cells. This measurement are sensitive to changes in airway diameter at all levels of the bronchiolar tree. An alternative approach would be to measure contractile activity of excised tracheal segments in vitro to determine the ability of a β-adrenergic agonist to relax airway smooth muscle that had been pre-contracted with methacholine. The major cellular sites of infection of the recombinant AAV vectors are in epithelial cells of the small and respiratory bronchioles as was found with infection of rats with recombinant adenoviruses.¹⁵³ In the experiments to determine the duration of the beneficial effect of β₂AR overexpression on lung function, the length of the experiment is set to 120 days. The observation is based on that respiratory epithelium has a turnover time (t^{½}) of approximately 100 to 120 days.⁹ After 120 days following infection, the beneficial effect of β₂AR overexpression is reduced by approximately 50%. This estimation assumes integration of the transgene into the host cell genome and that a significant population of epithelial stem cells is not transduced.

In situ hybridization is used to determine whether airway epithelial cells of recombinant AAV vector infected rats express the β₂AR transcription cassette. Anti-sense oligonucleotides directed against unique sequences in the cassette are used as probes in lung sections prepared from four mock-infected animals and four animals infected with d13-94/β₂AR/Neo^{SV40}. Detection of β₂AR transcription cassette MRNA will indicate that the transgene is being expressed.

Effect of β₂AR Overexpression on Lung Function

Sensitized Brown-Norway rats (three groups, five rats in each group) are infected with one of the following recombinant AAV vectors: d13-94/Neo^{SV40}, d13-94/β₂AR/Neo^{SV40} or d16-95/β₂AR/Neo^{SV40}. d13-94/Neo^{SV40} does not contain a β₂AR transcription cassette, d13-94/β₂AR/Neo^{SV40} contains a D₂AR transcription cassette driven by the β₂AR promoter with a GRE present, and d16-95/β₂AR/Neo^{SV40} contains a β₂AR transcription cassette driven by the AAV P5 promoter. Seven days following infection with recombinant AAV, animals are challenged with ovalbumin. Animals are instrumented and the sensitivity to the β-agonist, (−)-isoproterenol (as measured by decreased airway resistance) is determined following exposure to methacholine. The results from this experiment shows the beneficial effect of the overexpression of β₂AR in airway epithelial cells on lung function as measured by changes in airway resistance following β-agonist treatment. Lungs from all experimental animals are removed and stored at −70° C. for analysis by either in situ hybridization or immunohistochemistry determine the extent of recombinant β₂AR gene expression.

Duration of β₂AR Expression on Lung Function

Sensitized Brown-Norway rats (three groups, 32 rats in each group) are infected with one of the following recombinant AAV vectors: d13-94/Neo^{SV40}, d13-94/β₂AR/Neo^{SV40} or d16-95/β₂AR/Neosv⁴o Either 1, 2, 7, 14, 30, 60, 90 or 120 days following infection with recombinant AAV, animals are challenged with ovalbumin. Animals are instrumented and the sensitivity to the β-agonist (−)-isoproterenol (as measured by decreased airway resistance) determined following exposure to methacholine. The results from this experiment determine the duration of the beneficial effect of β₂AR overexpression on lung function as measured by changes in airway resistance following β-adrenergic agonist treatment. Lungs from all experimental animals are removed and stored at −70° C. for analysis by either in situ hybridization or immunohistochemistry determine the extent of recombinant β₂AR gene expression.

Effect of Inducement of β₂AR Expression on Lung Function

Sensitized Brown-Norway rats are divided into six groups with four rats in each group. Group I are infected with d13-94/Neo^{SV40} and treated with AAV vehicle for 7 days. Group II are infected with d13-94/Neo^{SV40} and treated with daily subcutaneous injections of 1 mg/kg dexamethasone for 7 days. Group III are infected with d13-94/β₂AR/Neo^{SV40} and treated with vehicle for7 days. Group IV are infected with d13-94/β₂AR/Neo^{SV40} and treated with daily subcutaneous injections of 1 mg/kg dexamethasone for 7 days. Group V are infected with d16-95/β₂AR/Neo^{SV40} and treated with vehicle for 7 days. Group VI are infected d16-95/β₂AR/Neo^{SV40} and treated with daily subcutaneous injections of 1 mg/kg dexamnethasone for 7 days. Previously it has been shown that daily injections of 1 mg/kg dexamethasone result in an approximate doubling of lung β₂AR number in the rat.⁸⁷ After seven days, animals are challenged with ovalbumin. Animals are instrumented and the sensitivity to the β-agonist (−)-isoproterenol (as measured by decreased airway resistance) determined following exposure to methacholine. The results from this experiment determine the extent of inducibility of recombinant β₂AR expression and the effect on lung function as measured by changes in airway resistance following β-agonist treatment. Groups V and VI are included to distinguish between the anti-inflammatory properties of glucocorticoids versus their effects on increasing the expression of β₂AR gene driven by the inducible promoter. The effect of dexamethasone on the expression of the β₂AR gene whose expression is driven by the AAV P5 promoter, is evaluated. Therefore, an enhanced sensitivity to (−)-isoproterenol following methacholine administration provides a measurement of the effects of dexamethasone that are not directly due to enhanced transcription of the β₂AR transgene under control of the inducible promoter. Lungs from all experimental animals are removed and stored at −70° C. for analysis by either in situ hybridization or immunohistochemistry determine the extent of recombinant β₂AR gene expression.

Effect of Exogenous Glucocorticoid Withdrawal on Lung Function

Sensitized Brown-Norway rats are divided into six groups with four rats in each group. Group I are infected with d13-94/Neo^{SV40} and treated with the AAV vehicle for 14 days. Group II are infected with d13-94/Neo^{SV40} and treated with dexamnethasone for 14 days. Group III are infected with d13-94/Neo^{SV40}, treated with dexamethasone for 7 days and then are withdrawn from glucocorticoids and treated with vehicle for an additional 7 days. Group IV are infected with d13-94/β₂AR/Neo^{SV40} and treated with vehicle for 14 days. Group V are infected with d13-94/β₂AR/Neo^{SV40} and treated with dexamethasone for 14 days. Group VI are infected with d13-94/β₂AR/Neo^{SV40}, treated with dexamethasone for 7 days and then are withdrawn from glucocorticoids and treated with vehicle for an additional 7 days. Dexamethasone (1 mg/kg) or vehicle are administered via daily subcutaneous injections. After 14 days, animals are challenged with ovalbumin. Animals are instrumented and the sensitivity to the β-agonist (−)-isoproterenol (as measured by decreased airway resistance) determined following exposure to methacholine. The results from this experiment will determine the effect of exogenous glucocorticoid withdrawal on lung function as measured by changes in airway resistance following β-adrenergic agonist treatment. Lungs from all experimental animals are removed and stored at −70° C. for analysis by either in situ hybridization or immunohistochemistry determine the extent of recombinant β₂AR gene expression.

Measurement of Airway Response

The procedure used to measure changes in airway resistance following methacholine challenge and subsequent treatment with β₂-adrenergic agonist is based on the original procedure for studying the mechanical properties of the lungs of guinea pigs.³ This method has been adapted by many investigators to measure airway resistance in the rat lung.^{37,139,149} Fourteen days after sensitization, rats are anesthetized with urethane (700 mg/kg intraperitoneally). The trachea is accessed via a midline incision and intubated with thin-walled stainless steel tubing (6 cm long). The intubation tubing is connected to a heated pneumotach (Hans Rudolph 8340, Kansas City, Mo.) and ports of the pneumotach are connected with latex tubing to differential pressure transducers (SCXL-EB, SenSym, Milpitas, Calif.). The instrumented rat is paralyzed (0.3 mg/kg gallamine) and the lungs are ventilated artificially (Harvard Apparatus Model 683, South Natick, Mass.). Heart rate is obtained by attaching surface electrodes to the skin that are connected to an ECG amplifer. Syringes (1 cc and 3 cc) are used to provide a volume calibration of the flow signal and a manometer is used to calibrate airway pressure. All physiological data are recorded on-line (Data Acquisition/Analysis, MP100 Acknowledge 3.0, BioPac Systems, Inc., Santa Barbara, Calif.). After completion of surgical procedures, animals are allowed to stabilize for 30 min. Methacholine is either infused into the jugular vein (1 mg/ml) or administered to the airways via a Marquest hand-held updraft nebulizer. The bronchodilatory effects of the β-adrenergic agonist (−)-isoproterenol are then evaluated after bronchoconstriction in response to methacholine reaches a steady-state level. Isoproterenol is administered either via the jugular vein or the nebulizer.

Construction of a Glucocorticoid-inducible β₂AR transgene for introduction into Airway Epithelium by AAV

Alternative constructs that are useful in the present invention are constructs containing inducible promoters that allow the control of the expression of the β₂AR gene in the target cells in the body of the subject. The following experiments disclose the preparation of a recombinant AAV that includes the coding region of the β₂AR gene and whose expression in epithelial cells is controlled by glucocorticoids. The expression is evaluated in SPOC1 cells in vitro. The optimum expression levels of the β₂AR gene may be increased by the modification of the promoter and the glucocorticoid response element.

First, corticosteroids are frequently used to treat asthmatic patients. This is done principally to control the inflammatory component of asthma. Therefore, expression of the transgene can be controlled by a therapeutic agent that most asthmatic patients already use. Second, glucocorticoids increase the rate of transcription of several genes including the β₂AR.⁵ This aspect of glucocorticoid action is considered in the design of the optimal β₂AR transgene for functional testing in airway epithelial cells in vitro and in vivo. Classically, glucocorticoids exert their effects by binding to a cytoplasmic glucocorticoid receptor causing the release of an associated 90 kDa heat shock protein and thereby allowing translocation of the receptor to the nucleus. Within the nucleus, glucocorticoid receptors form dimers that bind to DNA within steroid-responsive genes at consensus sequences called glucocorticoid response elements. This interaction changes the rate of transcription of the gene, most often resulting in induction of transcription, but in some cases gene expression can be repressed. The present inventors have identified the core GRE in the rat β₂AR gene as it functions in the HepG2 cell line as discussed below. Based on this work and other evidence, the expression of the rat β₂AR gene is induced by glucocorticoids. In these studies, the SPOC1 cell line is used to functionally characterize the cis-acting elements in the β₂AR gene that are involved in glucocorticoid induction. Glucocorticoid receptors bind to the consensus sequence GGTACAnnnTGTTCT (where n is any nucleotide). In some instances this may be a straight-forward interaction in which the receptor dimer bound to the GRE then interacts with basal transcription factors⁶⁷ or other DNA-binding proteins^{126,127} resulting in enhanced transcription of the target gene. However, in many cases the interactions are more complex. At composite GREs, the hormone receptor complex interacts with both specific DNA sequences and other transcription factors to regulate transcription of the target gene.^{31,47,91} Some transcription factor binding elements that interact with glucocorticoid response elements include those for activating protein-1,³ C/EBP⁵⁶ and hepatic nuclear factor 3 (HNF3). 148 Widely spaced glucocorticoid response elements have been shown to function in tandem to induce expression of the tryptophan oxygenase gene.²⁷ The data obtained from transient expression of β₂AR-luciferase fusion genes in HepG2 cells indicates complex regulation of β₂AR gene expression by glucocorticoids that appears to involve other as yet unidentified genetic elements.

Cloning of the Rat β₂AR Gene 5′-Flanking DNA

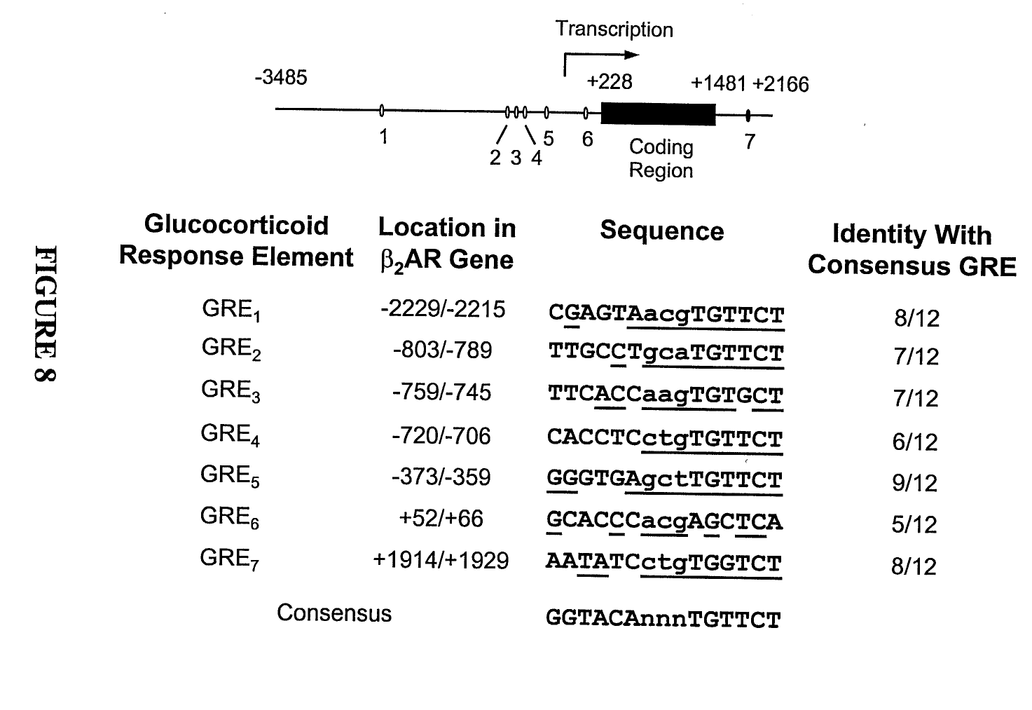

To study transcriptional regulation of β₂AR gene expression, a rat β₂AR genomic clone was inserted into the EcoRi site of X phage Charon 4A from Dr. P. Buckland, University of Wales. The clone includes the 4190 bp section submitted to the Genebank/EMBL database,¹⁶ plus an additional unsequenced section of aproximately 1400 bp in the 5′-flanking region. The portion sequenced by Buckland¹⁶ includes 2251 bp of the 5′-flanking region, 1256 bp coding region, and 682 bp of the 3′-untranslated region. A 4200 bp section of the original clone was subcloned that encodes nucleotides −3912 to +322 (relative to the start of transcription) into the EcoRI-KpnI sites of plasmid pGEM7zF(−) (Promega, Madison, Wis.). Independently, Jiang and Kunos⁷⁰ and McGraw et al.⁸⁸ discovered that a 2062 bp PstI-PstI fragment of the 5′-flanking region was entered incorrectly into the Genebank/EMBL database. The corrected sequence along with an additional −1 kb of 5′-flanking sequence in the NCBI nucleotide sequence database under the accession number U35448. With reversal of orientation of the PstI-PstI fragment, the similarity between the 5′-flanking regions of the rat and human genes becomes 74%, as determined by the BESTFIT program of the GCG Sequence Analysis Software Package. This degree of identity is similar to the 73% reported between the human and mouse β₂AR genes.^{39,95} Reversing the sequence conserves putative promoter elements that are found in the β₂AR genes of other species. A reverse complement CAAT box in the human and hamster genes is conserved in the rat, as are two TATA box-like sequences. From primer extension analysis, the start of transcription is −220⁷¹ relative to the first nucleotide of the initiator ATG for the receptor open reading frame. FIG. 8A provides a schematic representation of the β₂-AR gene.

Computer analysis of the known sequence of the rat β₂AR receptor gene yielded seven potential glucocorticoid regulatory elements (FIG. 8B). Six of the potential GREs are located upstream of the receptor open reading frame while the seventh GRE is located in the 3′-flanking region of the gene. Sequence comparisons were made between the seven putative β₂AR gene GREs and the distal GRE upstream of the mouse mammary tumor virus (MMTV) promoter.¹⁰³ The MMTV GRE, which contains the core GRE sequence TGTTCT, binds glucocorticoid receptor and is necessary for hormone responsiveness.¹²⁴ Studies show the putative GRE downstream of the receptor open reading frame was found to be nonfunctional. Attention was focused on the six GRE-like elements in the 5′-flanking region of the gene.

Transient Transfections of β₂AR Promoter Truncations

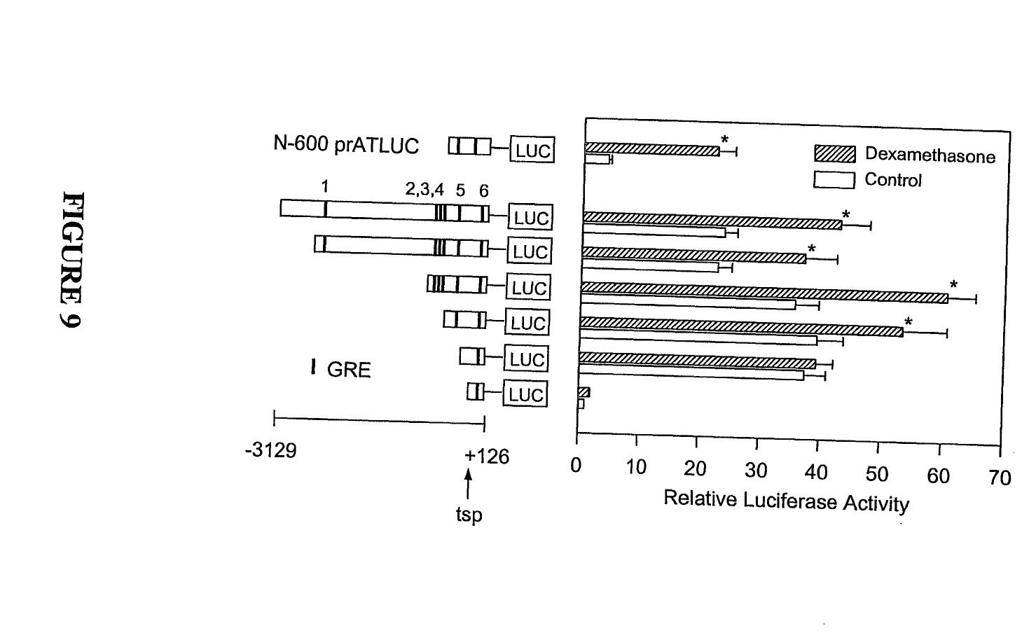

In order to determine whether the six putative GREs were functional, six β₂AR-luciferase fusion gene deletion mutants were generated. Among them, pβ₂AR(−3129/+126) and pβ₂AR(−2552/+126) contain all six putative GREs, pβ₂AR(−1115/+126) contains the proximal five putative GREs, pβ₂AR(−643/+126) contains GRE₅ and GRE₆, and pβ₂AR(152/+126) and pβ₂AR(−62/+126) contain only the most proximal GRE. For experiments in which dexamethasone-stimulated promoter activity in different 5′-deletion constructs was A) tested, subconfluent cells in DMEM with 10% fetal bovine serum that had been stripped of steroids were transfected with 0.38 μmoles of the β₂AR-luciferase fusion genes, 2 μg pRSVβ-gal, 1 μg pRShGRα and pGEM-7Zf(−) to adjust the amount of total DNA per dish to 8.33 μg. To test the effect of dexamethasone on expression of the truncated β₂AR-luciferase fusion genes, HepG2 cells transfected with each of the six fusion genes were incubated with either vehicle or 0.1 μM dexamethasone for 8 hrs, and luciferase activity was determined. The HepG2 human liver cell line has previously been used to study glucocorticoid regulation of angiotensinogen gene expression.¹³ HepG2 cells are deficient in functional glucocortocid receptors.¹² By this deficiency, the role of glucocorticoids in regulating β₂AR gene expression by cotransfection with pRShGRα, a glucocorticoid receptor-encoding expression plasmid, is investigated. The activity of pRSVβ-gal was used to correct for differences in transfection efficiency between individual experiments. Results from preliminary experiments indicated that 8 hours was the optimal time to observe dexamethasone responsiveness since cell viability decreased with longer exposures to dexamethasone (data not shown). The data shown in FIG. 9 depict the results of experiments in which progressively truncated β₂AR-luciferase fusion genes transiently transfected into HepG2 cells were tested for dexamethasone responsiveness. Approximate two-fold induction with 0.1 μM dexamethasone over that in the absence of added glucocorticoid was observed with pβ₂AR(−3129/+126), pβ₂AR(−2552/+126), and β₂AR(−1115/+126). This level of induction of luciferase expression is similar to the fold induction in β₂AR levels that has been observed in the lung following injection of glucocorticoids,^{19,84,87} and with addition of glucocorticoids to cultured cells. ^{19,28,46,104,140,157} With pβ₂AR(−643/+126), the induction observed with 0.1 μM dexamethasone was approximately 1.5-fold. With pβ₂AR(−152/+126) and pβ₂AR(−62/+126), luciferase activity with the addition of 0.1 μM dexamethsone was not significantly increased over that in the absence of added glucocorticoid. As a positive control, N-600 prATLUC, a fusion gene containing a segment of the rat angiotensinogen gene with two functional GREs coupled to a luciferase-encoding gene, was used. In the presence of 0.1 μM dexamethasone, expression of N-600 prATLUC was increased approximately 8- to 10-fold, consistent with the level of induction by glucocorticoid previously demonstrated with this fusion gene.¹³ Taken together, the data indicate that of the six putative glucocorticoid response elements in the 5′-flanking region of the β₂AR gene, only GRE₅ appears to be functional.

Truncation from the −2552 position to the −1115 position produced an increase in basal activity in the absence of added dexamethasone (FIG. 9). This observed increase of basal activity in the shorter constructs is not unusual in studies of this type. One interpretation of this result is the presence of negative regulatory elements in the truncated region, in this case between −2552 and −1115. Alternatively, the difference in activity may also be related to the proximity of plasmid vector sequences to regulatory elements in the β₂AR gene sequence.

Transient Transfections Using a β₂-Adrenergic Receptor-Luciferase Fusion Gene with Mutated GRE₅

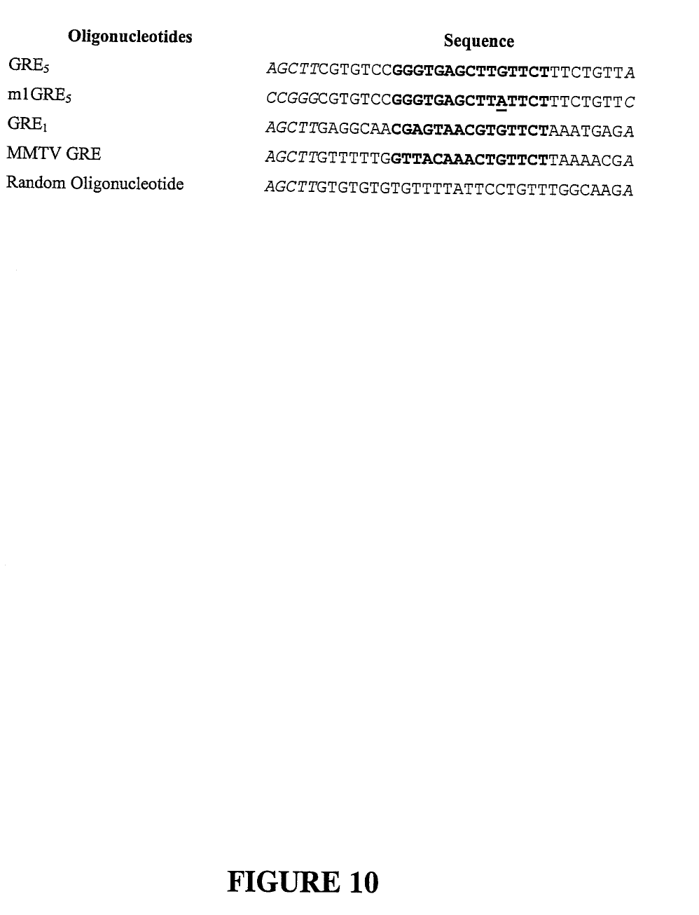

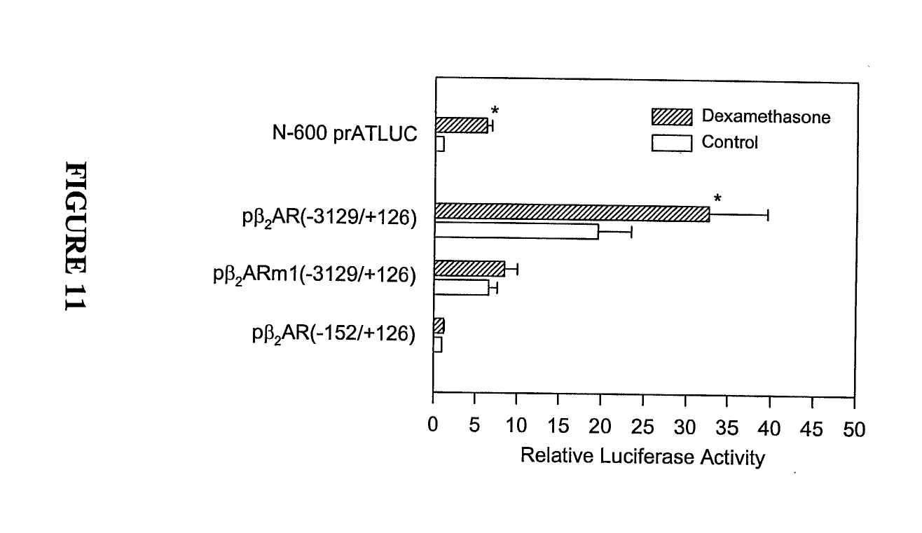

To test further the involvement of GRE₅ in glucocorticoid regulation of β₂AR expression, a plasmid pβ₂ARm1(−3129/+126) was constructed that had been mutated at position +6 of GRE5 (GGGTGAGCTGTTCT→GGGTGAGCTATTCT). This mutation, the same base change in oligonucleotide m1GRE₅ (FIG. 10), is essential for glucocorticoid inducibility of a MMTV GRE. ¹⁰³ The results demonstrate loss of glucocorticoid inducibility using pβ₂ARm1(−3129/+126) (FIG. 11). Interestingly, in the absence of added dexamethasone, activity of pβ₂ARm1(−3129/+126) was markedly lower than that of pβ₂AR(−3129/+126) (FIG. 11). It appears that basal expression of pβ₂AR(−3129/+126) in HepG2 cells that are over-expressing glucocorticoid receptor is relatively high despite removal of glucocorticoids from serum by charcoal stripping. Forty-eight hours prior to transfection, the HepG2 cells are switched to charcoal-stripped serum. Alternatively, GRE₅ contributes to basal activity of the β₂AR gene promoter.

Transient Transfections Using Putative GREs Fused to a Heterologous Promoter

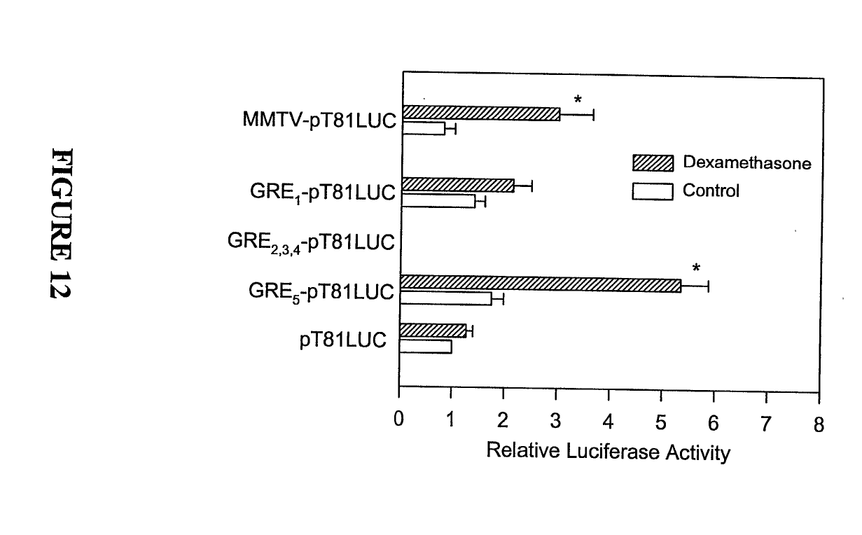

To further examine the functionality of the putative glucocorticoid response elements, fragments containing either GRE₁, GRE₅, or GRE₂, GRE₃ and GRE₄ together were fused to pT81LUC, a luciferase expression plasmid driven by a minimal thymidine kinase promoter.¹⁰² Sequences of the double stranded oligonucleotides containing GRE₁, GRE₅ and the MMTV GRE are shown in FIG. 10. As a positive control, a MMTV-pT81LUC fusion gene was prepared which contains a previously demonstrated functional GRE.^{17,80} An approximate 4-fold induction was observed with 0.1 μM dexamethasone using the MMTV-pT81LUC fusion gene (FIG. 12). Activity of GRE₅-pT81LUC was induced 3.2-fold in the presence of 0.1 μM dexamethasone (FIG. 12), a value that was higher than that observed with any of the β₂AR-luciferase fusion genes that included GRE₅. Activity of GRE₁-pT81LUC was not induced by 0.1 μM dexamethasone (FIG. 12). Transfection of HepG2 cells with segment −831 to −708, which contains GRE₂, GRE₃, and GRE₄, fused to pT81LUC resulted in luciferase activity in either vehicle or 0.1 μM dexamethasone treated cells that was below that observed in mock-transfected cells. Of interest within this experiment, is the observation that GRE₅, when fused to pT81LUC and transiently transfected into HepG2 cells, resulted in 3.2-fold induction of activity. This level of fold-induction was similar to that obtained with MMTVsequence fused to pT81LUC. These results indicate either that the β₂AR promoter is a relatively weak substrate for GRE enhancer activity or that other segments of the β₂AR gene missing from our reporter gene constructs are necessary for full induction by glucocorticoids.

In Vitro Characterization of the Interactions of the Glucocorticoid Receptor and Other Putative Transcription Factors with β₂AR Gene DNA: Electrophoretic Mobility Shift Assays and Supershift Assays

Electrophoretic mobility shift assays and supershift assays are used to detect the presence of specific DNA binding proteins in SPOC1 cells treated with and without 0.1 μM dexamethasone. Sense and anti-sense oligonucleotides (^{˜}35-50 nucleotides) are commercially synthesized (Bio-Synthesis, Inc., Lewisville, Tex.). Complimentary oligonucleotides in equimolar amounts are heated to 100° C. and cooled overnight to 25° C., aliquoted and stored at −20° C. prior to use. Labeled probes are prepared by either end-labeling with [α-³²P]ATP using T4 polynucleotide kinase or by filling in 5′-overhangs with a [α-³²P]dNTP using Klenow. Binding reactions are performed in a 20 μl volume that includes approximately 20,000 cpm labeled probe, 6 to 12 μg nuclear extract, 20 mM HEPES, pH 7.9, 60 mM KCl, 5 mM MgCl₂, 2 mM dithiothreitol, 10% glycerol, 200 ng polydI.dC (to reduce nonspecific binding), 1 μg bovine serum albumin, and unlabeled competitor oligonucleotides. After incubation for 30 minutes at room temperature, the reactions are loaded onto 6% non-denaturing polyacrylamide gels in 1×TBE (25 mM Tris, 25 mM boric acid, 0.5 mM EDTA) to separate protein-DNA complexes from free radiolabeled probe. Gels are dried and subjected to autoradiography. The electrophoretic mobility of DNA that specifically binds nuclear proteins are retarded compared to the mobility of DNA that is not complexed to protein. Observed DNA-protein complexes are characterized for specificity by competition with varying amounts of other DNA fragments; either unlabeled probe or other double-stranded oligonucleotides with similar sequences. In competition assays, increasing amounts of unlabeled double-stranded oligonucleotides that binds the transcription factor (e.g., the glucocorticoid receptor) of interest competes with binding of labeled probe causing loss of binding signal as the concentration of the unlabeled probe is increased. Competition with a unlabeled double-stranded oligonucleotide with little sequence identity with the labeled probe will not change the intensity of the binding signal. To further delineate the proteins that bind to labeled probes, supershift assays are performed. For these studies, commercially obtained antibodies specific for the putative transcription factor (e.g., glucocorticoid receptor, API, etc.) are added to the reaction mixture described above for either 16 hrs at 4° C. or 50 min at 30° C. prior to addition of the radiolabeled double-stranded oligonucleotide. When a putative transcription factor is present in the DNA-protein complex, then the electrophoretic mobility of the complex are altered (usually retarded) due to binding of the antibody resulting in a “supershifted” profile. These results confirm the identity of the specific trans-acting factor(s) involved. For electrophoretic mobility shift assays and supershift assays, nuclear extracts are prepared from at least three different cell culture plates and at least three different experiments performed.

Electrophoretic Mobility Shift Assays Using Nuclear Extracts Prepared from Dexamethasone-Treated HepG2 Cells

Cells are cultured in the presence and absence of 0.1 μM dexamethasone for 8 hrs. Nuclear extracts from both groups of cells are prepared using the method of Andrews and Faller.⁴ The protocol is a micro-preparation procedure that allows rapid extraction of DNA-binding proteins from small numbers of cells. Except where otherwise noted, centrifugations are done in an Eppendorf Model 5415C microfuge at maximum speed at room temperature. Approximately 10⁶ to 10⁷ SPOC1 cells from the control and dexamethasone treated groups are scraped into 1.5 ml ice-cold phosphate buffered saline, pH 7.4 and pelleted. Cells are re-suspended in 400 μl Buffer A (10 mM HEPES-KOH), pH 7.9, 1.5 mM MgCl₂, 10 mM KCl, 0.5 mM dithiothreitol, 0.2 mM phenylmethylsulfonylfluoride) at 4° C. Cells were allowed to swell for 10 min, then vortexed for 10 sec, centrifuged for 10 sec, and the supernatant discarded. The pellet is resuspended in 20 to 50 μl of Buffer C (20 mM HEPES-KOH, pH 7.9, 25% glycerol, 420 mM NaCl, 1.5 mM MgCl₂, 0.2 mM EDTA, 0.5 mM dithiothreitol, 0.2 mM phenylmethylsulfonylfluoride) at 4° C. and incubated for 20 min. Cellular debris is removed by centrifugation for 2 min at 4° C. and the supernatant containing DNA binding proteins is stored at −70° C. Nuclear extract protein concentrations are determined¹¹ using bovine serum albumin as the standard. The yield of this procedure is approximately 50-75 μg of protein per 10⁶ cells, a sufficient quantity of nuclear protein for 10-12 lanes in a single electrophoretic mobility shift assay.

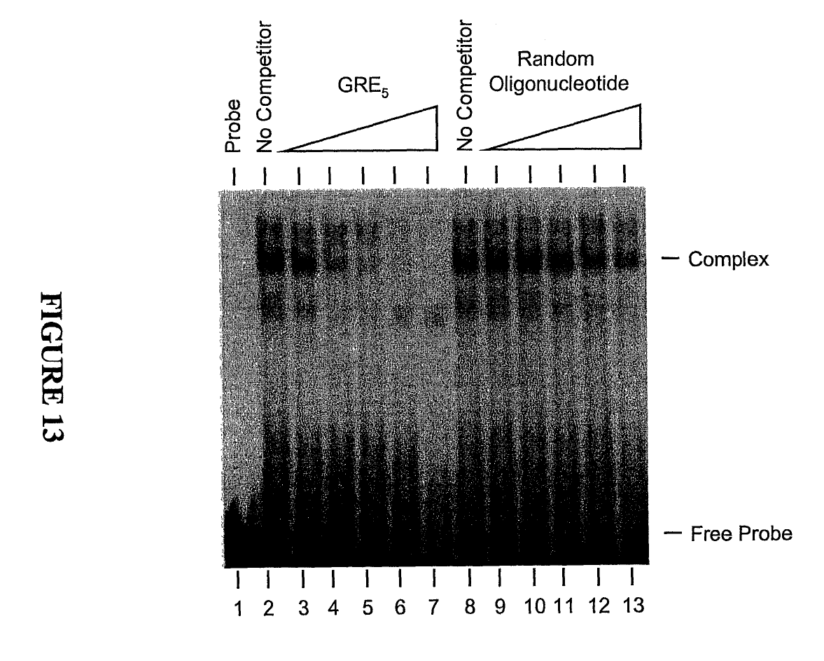

To determine if nuclear transcription factors could indeed bind to GRE₅ from the rat β₂AR gene, electrophoretic mobility shift assays were performed using as the probe a 35 bp double stranded oligonucleotide that includes GRE, and flanking sequence, and nuclear extracts prepared from HepG2 cells that had been treated with 0.1 μM dexamethasone for 8 hrs. In preliminary experiments in which increasing amounts of HepG2 cell nuclear extract was added to radiolabeled GRE₅ probe, it was determined that 6 μg of nuclear extract resulted in optimum levels of shifted product (data not shown). The results of a representative experiment is shown which depicts an electrophoretic mobility shift assay in which the specificity of the interaction of HepG2 cell nuclear proteins with GRE₅ was assessed by competition analysis (FIG. 13). Radiolabeled GRE₅ incubated with HepG2 cell nuclear extract resulted in a prominent shifted band (FIG. 13). Addition of increasing concentrations of unlabeled GRE₅ displaced radiolabeled GRE₅ in a dosedependent manner whereas the unlabeled oligonucleotide designated random oligonucleotide did not suppress radiolabeled GRE₅ binding (FIG. 13).

Electrophoretic Mobility Shift Assays Using Recombinant Human Glucocorticoid Receptor

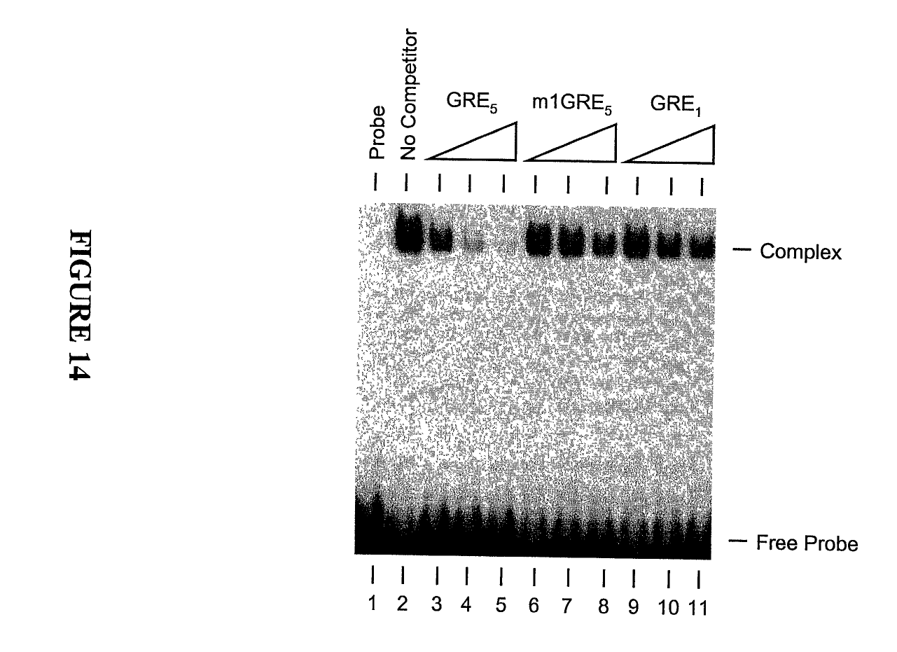

To further characterize the ability of GRE₅ to bind glucocorticoid receptor in vitro, we performed electrophoretic mobility shift assays with recombinant human glucocorticoid receptor and serial dilutions of various competitor oligonucleotides was performed. Radiolabeled GRE₅ incubated with recombinant human glucocorticoid receptor resulted in a single shifted band (FIG. 14). Addition of increasing concentrations of unlabeled GRE₅ displaced radiolabeled GRE₅ in a dose-dependent manner (FIG. 14). In contrast, increasing concentrations of either unlabeled GRE, or unlabeled mlGRE₅, with a single nucleotide change in the core sequence of GRE₅, did not compete with radiolabeled GRE₅ for binding to recombinant human glucocorticoid receptor (FIG. 14). This change (GSA in position +6 of the GRE) had previously been shown to result in the complete loss of glucocorticoid inducibility of a MMTV GRE fused to a luciferase reporter gene.¹⁰³

Transient Transfection Experiments to localize the segment of the β₂AR gene that contains essential elements for basal and glucocorticoid-stimulated expression

SPOC1 cells are cultured in monolayers as described above. Twenty-four hours prior to transfection, cells are split into 60 mm culture dishes containing 2 ml of growth media. Cell confluency are ^{˜}50% at the time of transfection. In a given experiment, cells are transfected using Lipofectamine (Gibco-BRL, Gaithersburg, Md.) in triplicate with either pGL3-Basic (a negative control plasmid that has no promoter or enhancer sequences), or pGL3-Control (a positive control plasmid with promoter and enhancer activity driven by the SV40 viral promoter), or the β₂AR-Luciferase fusion genes. All cells are co-transfected with pRSVP-gal (Promega, Madison, Wis.). pRSVβ₂-gal encodes β-galactosidase which is used to adjust for transfection efficiency. Cells in 60 mm plates are co-transfected with 2.4 μg of DNA, which includes a particular β₂AR-Luciferase fusion gene (the amount of DNA transfected will depend upon the molecular weight of the construct), 0.4 μg pRSVβ-ga1, and pGEM7Zf(−) as carrier DNA. Cells are then incubated overnight in growth media. After the incubation, fresh media are applied, and treatment cells are stimulated with 0.1 μM dexamethasone; control cells will receive the vehicle. Cells are incubated for an additional 8 hrs at which time cell lysates are harvested using Lysis Buffer (Promega, Madison, Wis.). Cell lysates are assayed for luciferase activity using the ProMega luciferase assay system (Madison, Wis.) and β-galactosidase using the Galacto-Light system (TROPIX, Bedford, Mass.) using a Monolight Model 2010 Luminometer. To adjust for transfection efficiency, firefly luciferase activity are normalized to β-galactosidase activity. Firefly luciferase activity, corrected for β-galactosidase activity, for each construct are compared to values obtained with pGL3-Control, a luciferase reporter gene driven by the SV40 promoter (a positive control which should give high firefly luciferase activity in transfected <5 cells) and pGL3-Basic (a control lacking a promoter which should result in low firefly luciferase activity in transfected cells). These experiments identify the minimal promoter that directs β₂AR gene expression in SPOC1 cells, the glucocorticoid responsive element and other enhancer-like and/or repressor-like mechanisms that regulate β₂AR gene expression. Within each experiment, constructs are tested in triplicate and each experiment is repeated four to five times.

In Vitro Characterization of the Interactions of the Glucocorticoid Receptor and Other Putative Transcription Factors with β₂AR Gene DNA: DNase I Footprinting

In order to establish the identities of the factors interacting in vivo to glucocorticoid responsiveness to the β₂AR gene, a comparative analysis of the in vitro footprints obtained with DNase I are performed. These experiments are conducted in order to detect possible transcription factor interaction with β₂AR gene DNA. DNase I footprinting are performed using previously described methods.⁵⁰ Nuclear extracts are prepared from untreated and 0.1 μM dexamethasone-treated SPOC1 cells as described for electrophoretic mobility shift assays. Target DNA (either restriction fragments<120 bp or complementary oligonucleotides) that includes the GRE and possible interacting cis-elements are either endlabeled with [γ-³²P]ATP using T4 polynucleotide kinase or by filling in 5′-overhangs with a [α-³²P]dNTP using Klenow. Labeled DNA fragments are incubated with nuclear extracts prepared from untreated and dexamethasone-treated SPOC1 cells in a binding buffer (10 mM Tris-HCl, pH 7.5, 50 mM NaCl, 0.05% Nonidet P-40, 1 mM EDTA, 1 mM DTT and 10% glycerol) for 30 min at room temperature. The reaction mixture are digested with DNase I. Digestion is stopped with a solution containing 30 mM EDTA and 0.15% SDS. Exact conditions (amount of nuclear extract, DNase I concentration, digestion time, etc.) are optimized. Following phenol-chloroform extraction, pellets are denatured and resolved on 6% polyacrylamide-urea sequencing gels. Protected regions are visualized by autoradiography to allow identification of specific bases involved in transcription factor binding. Identification of the specific nucleotide sequences involved allows the determination if more than one region of the gene is involved in transcription factor binding. For both untreated and dexamethasone-treated cells, nuclear extracts are prepared from at least three different cell culture plates and used in DNase I footprinting analysis.

Deletions and Mutagenesis

In order to confirm the functional significance of the identified cis-elements involved in glucocorticoid regulation of β₂AR gene expression, mutations are introduced into putative regulatory sequences using the method of Landt et al.¹⁷ Although changing sequence can change secondary structure which can in turn produce an effect, mutagenesis is a standard and time tested method for determining the role of potential elements in transcriptional and other genetic events. Only short sequences are changed which should minimize effects on secondary structure. Mutagenic fragments are created with a two-step PCR procedure that includes an intermediate purification step. In the first PCR step, a 5′-universal primer and a 3′-mutagenic primer are used to generate a double-stranded mutated fragment. The PCR products are separated from the primers on 1% agarose gels, and the fragments of interest are eluted. In the second PCR step, the purified product from the first PCR reaction is used as the 5′-mutagenic primer in combination with a second 3′-universal primer to generate the fmal product. The product of the second PCR step is then digested with appropriate restriction endonucleases and is subcloned into an appropriate plasmid vector. Primer design and PCR conditions are determined for the sequence to be mutagenized. However, the 3′- and 5′-universal primers will incorporate restriction sites that facilitate rapid subcloning of mutagenized fragments. Sequences are verified using the dideoxynucleotide chain termination method¹²³ and Sequenase (United States Biochemical Co., Cleveland, Ohio). Functional capabilities of the mutagenized fragments are determined in transiently transfected SPOC1 cells treated with 0.1 μM dexamethasone and with electrophoretic mobility shift assays with nuclear extracts prepared from dexamethasone-treated SPOC1 cells as described above.

Identification of Functional β₂AR Coupled to Adenvlvl Cyclase in the Rat Tracheal Epithelial Cell Line SPOC1

Cell Culture: Tracheal Epithelial Cell Line SPOC1

SPOC1 cells are a continuous cell line spontaneously derived from secondary rat tracheal epithelial cultures.³² They are nontumorigenic in nude mice, maintain a diploid karyotype, and when implanted into denuded rat tracheas form a stratified squamous epithelium that eventually forms glandlike invaginations into the surrounding lamina propria.³² The SPOC1 cells utilized in these experiments were originally provided by Dr. Patrice Ferriola (Chemical Industry Institute of Toxicology, Research Triangle Park, N.C.). SPOC1 cells are grown in Dulbecco's Modified Eagles Medium (DMEM) and Ham's F-12 Medium (1:1) supplemented with 5% fetal bovine serum, 0.1 μg/ml hydrocortisone, 5 μg/ml insulin, 100 U/ml penicillin G and 100 μg/ml streptomycin as previously described.³² In experiments in which the effect of dexamethasone is tested on the expression of β₂AR and coupling to adenylyl cyclase, hydrocortisone is removed from the media 72 hrs prior to the experiment.

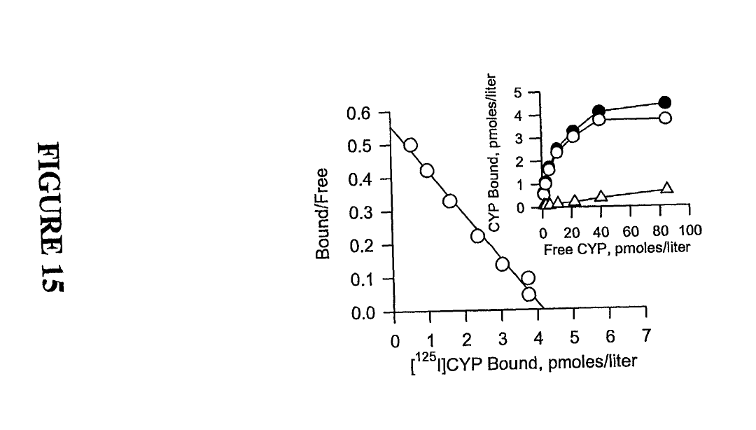

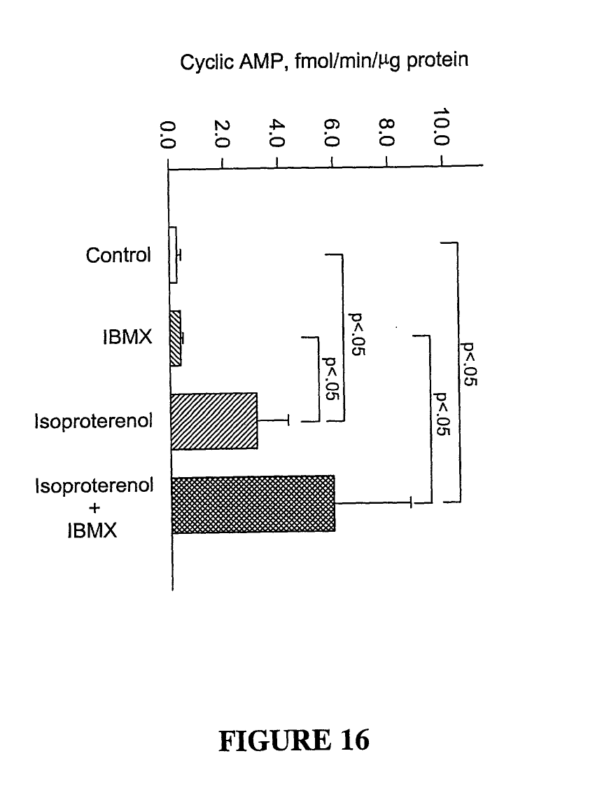

SPOC1 cells are useful to determine the β₂AR function in lung epithelium under defmed conditions. In preliminary experiments, β₂AR numbers are determined by radioligand assays using [¹²⁵I]cyanoiodopindolol ([¹²⁵I]CYP). [¹²⁵I]CYP binding to SPOC1 cell membranes was to a single, saturable site that displayed high affinity as demonstrated in the representative Scatchard plot (FIG. 15). From this experiment, the [¹²⁵I]CYP dissociation constant was 10 pM and the binding site concentration was 60 fmol/mg protein. From competition experiments with [¹²⁵I]CYP, the rank order of potency of adrenergic agonists was isoproterenol (Kᵢ=0.07±0.04 μM)<epinephrine (Kᵢ=0.5±0.2 μM)<norepinephrine (Kᵢ=11±5 μM) (data not shown). Thus, the [¹²⁵I]CYP the binding site has pharmacological characteristics of the β₂AR subtype. To determine whether the β₂AR in SPOC1 cells is functionally coupled through β₂AR to adenylyl cyclase, cyclic AMP accumulation was measured in cells treated with isoproterenol, a β-adrenergic agonist. Preliminary experiments were conducted in order to determine the effect of the phosphodiesterase inhibitor isobutylmethylxanthine (IBMX) on isoproterenol-stimulated cyclic AMP accumulation (FIG. 16). Cyclic AMP production in control (vehicle-treated) cells was 0.27±0.08 fmol/min/mg protein. The addition of 100 μM IBMX resulted in a small, but statistically insignificant increase in cyclic AMP accumulation to 0.40±0.04 fmol/min/mg protein. In the presence of 10 μM isoproterenol, cyclic AMP production was significantly (p<0.05) increased over that of control cells to 3.17±0.57 fmol/min/mg protein. Cyclic AMP production was further increased to 5.95±1.4 fmol/min/mg protein in the presence of both 100 μM IBMX and 10 μM isoproterenol. The results of these experiments demonstrate that SPOC1 cells express the β₂AR subtype and that the receptor is functionally coupled to adenylyl cyclase and generation of intracellular cyclic AMP. Airway epithelial cells in a number of mammalian species have been shown to express the β₂AR subtype.^{30,72,101,125} Therefore, SPOC1 cells have retained an important signal transduction pathway in the regulation of lung epithelial cell biology.

Transient Transfections of SPOC1 Cells

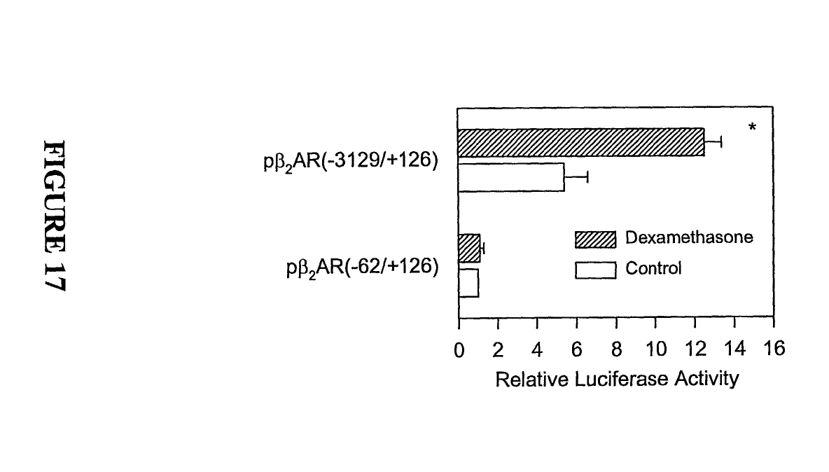

Preliminary experiments show the optimum methodology for transiently transfecting SPOC1 cells. Calcium phosphate co-precipitation and a cationic liposome-mediated method are compared. Lipofectamine (Gibco-BRL, Gaithersburg, Md.) was consistently superior to calcium phosphate co-precipitation for SPOC1 cell transfections as judged by expression levels of pβ₂AR(−3129/+126) (data not shown). Cells were transiently transfected with a total of 2 μg of DNA which includes pβ₂AR(−3129/+126), a positive control plasmid pRVL-SV40 that expresses Renilla luciferase under the direction of the SV40 viral promoter, and pGEM 7Zf(−) DNA. Renilla luciferase activity was used to correct for differences in transfection efficiencies between individual experiments. Luciferase activity is measured in cell lysates using the Dual Luciferase Assay Kit (Promega, Madison, Wis.), which allows measurement of Firefly and Renilla luciferase activity in rapid succession in a single tube. Firefly luciferase activity, which arises from β₂AR(−3129/+126), corrected for Renilla luciferase activity was linear in SPOC1 cells transfected with increasing amounts of pβ₂AR(−3129/+126) (data not shown). In prelimrinary experiments, the effect of dexamethasone on expression of pβ₂AR(−3129/+126) in SPOC1 cells is tested. Dexamethasone was added for 8 hrs prior to harvesting cell lysates for determination of luciferase activity. Similar to results obtained with HepG2 cells, 0.1 μM dexamethasone produced an approximate 2-fold induction in luciferase activity in transiently transfected SPOC1 cells (FIG. 17). There was no induction of luciferase activity by dexamethasone in cells that had been transiently transfected with pβ₂AR(−62/+126). These results indicate that the SPOC1 cell line is a suitable model for studying transcriptional regulation of β₂AR gene expression by glucocorticoids and for testing expression and regulation of β₂AR transgenes carried in recombinant viruses.

Reference is made to a number of publications and patents in the present application. There mentioned documents are incorporated in their entirety by reference.

Literature Cited

1. Alton E. W. F. W. and D. M. Geddes, Gene Therap. 2:88-95, 1995.

2. Alwine, J. C., Molec. Cell. Biol. 5:1034-1042, 1985.

3. Amdur, M. O. and J. Mead, Amer. J. Physiol. 192:364-368, 1958.

4. Andrews, N. C. and D. V. Faller, Nuc. Acids Res. 19: 2499, 1991.

5. Barnes, P. J., Amer. J. Resp. Ctit. Care Med. 152:838-860, 1995.

6. Barnes, P. J., J. Int. Med. 231:453-461, 1992.

7. Barnes, P. J., N. Engl. J. Med. 332:868-875, 1995.

8. Barnes, P. J. et al., Nature 299:444-447, 1983.

9. Blenkinsopp, W. K., Exp. Cell Res. 46:144-154, 1967.

10. Blodgett, F. M. et al., New Eng. J. Med. 254:636-641, 1956.

11. Bradford, M. M., Anal. Biochem. 72: 248-254, 1976.

12. Brasier, A. R., et al. Molec. Endocrinol. 4:1921-1933, 1990.

13. Brasier, A. R. et al., Molec. Endocrinol. 3:1022-1034, 1989.

14. Breeze, R. G. and E. B. Wheeldon, Amer. Rev. Respir. Dis. 116:705-777, 1977.

15. Brown, P. H. et al., Thorax 48:967-973, 1993.

16. Buckland, P. R. et al., Nucl Acids Res. 18:682, 1990.

17. Buetti, E. and B. Kuhnel, J. Mol. Biol. 190:379-389, 1986.

18. Chaturvedi, C. M. et al., Peptides 15:1179-1187, 1994.

19. Cheng, J. B. et al., Endocrinology 107:1646-1648, 1980.

20. Cheng, Y. -C. and W. H. Prusoff, Biochem. Pharmacol. 22: 3099-3108, 1973.

21. Chow, Y -H. et al., Proc. Natl. Acad. Sci. USA 94:14695-14700, 1997.

22. Church, M. K. and J. Hiroi, Br. J. Phannacol. 90:421-429, 1987.

23. Collins, S. et al., J. Biol. Chem. 263:9067-9070, 1988.

24. Coqueret, O. et al., Ann. NY Acad. Sci. 725:44-49, 1994.

25. Crane, J. et al., Brit. Med. J. 304:1307, 1992.

26. Crystal, R. G. Science 270:404-410, 1995.

27. Danesch, U. et al., EMBO J. 6: 625-630, 1987.

28. Dangel, V. et al., Biochem. J. 317:925-931, 1996.

29. Davis, C. and M. E. Conolly. Brit. J. Clin. Pharmacol. 10:417-423, 1980.

30. Davis, P. K. et al., Amer. J. Physiol. 258:C71-C76, 1990.

31. Diamond, M. I. et al., Science 249:1266-1272, 1990.

32. Doherty, M. M. et al., Amer. J. Respir. Cell Mol. Biol. 12:385-395, 1995.

33. Dulis, B. H. and T. B. Wilson, J. Biol. Chem. 225:1043-1048, 1980.

34. Eggleston, P. A., Pediatrics 99:729-730, 1997.

35. Eidelman, D. H. et al., Amer. Rev. Respir. Dis. 137:1033-1037, 1988.

36. Ellul-Micallef, R. and F. F. Fenech, Lancet 2:1269-1271, 1975.

37. Elwood, W. et al., J. Allergy Clin. Immunol. 88:951-960, 1991.

38. Elwood, W. et al., J. Appl. Physiol. 75:279-284, 1993.

39. Emorine, L. J. et al., Proc. Natl. Acad. Sci. USA 84:6995-6999, 1987.

40. Executive summary: guidelines for the diagnosis and management of asthma. National Asthma Education Program Expert Panel Report. U.S. Department of Health and Human Services, Public Health Services, National Institutes of Health, 1992.

41. Ferkol, T. et al., J. Clin. Invest. 95:493-502, 1995.

42. Flavahan, N. A. et al., J. Appl. Physiol. 58:834-838, 1985.

43. Flotte, T. R. et al., Amer. J. Respir. Cell Mol. Biol. 7:349-356, 1992.

44. Flotte, T. R. et al., Proc. Natl. Acad. Sci. USA 90:10613-10617, 1993.

45. Flotte, T. R. et al., J. Biol. Chem. 268:3781-3790, 1993.

46. Fraser, C. M., and J. C. Venter. Biochem. Biophys. Res. Comm. 94:390-397, 1980.

47. Frenkel, B. et al., Proc. Natl. Acad. Sci. USA 91:10923-10927, 1994.

48. Funder, J. W. Science 259:1132-1133, 1993.

49. Furu, K. et al., J. Steroid Biochem. 28:587-591, 1987.

50. Galas, D. J. and A. Schmitz. Nucl. Acids Res. 5:3157-3170, 1978.

51. Gazit, G. et al., Cancer Res. 55:1660-1663, 1995.

52. Geddes, B. A. et al., Amer. Rev. Respir. Dis. 110:420-427, 1974.

53. Geddes, D. M. Thorax 47:404-407, 1992.

54. Goldman, M. J. et al., Human Gene Therapy 6:839-851, 1995.

55. Grainger, J. et al., Thorax 46:105-111, 1991.

56. Grange, T. et al., Nucl. Acids Res. 19: 131-139, 1991.

57. Hadcock, J. R. and C. C. Malbon. J. Neurochem. 60:1-9, 1993.

58. Hadcock, J. R. and C. C. Malbon. Proc. Natl. Acad. Sci. USA 85:8415-8419, 1988.

59. Hadcock, J. R. et al., J. Biol. Chem. 264:19928-19933, 1989.

60. Halbert, C. L. et al., J. Virol. 71:5932-5941, 1997.

61. Hayashi, Y. et al., J. Biol. Chem. 269:23872-23875, 1994.

62. Hermonat, P. L., and N. Muzyczka. Proc. Natl. Acad. Sci. USA 81:6466-6470, 1984.

63. Hermonat, P. L. et al., FEBS Lett. 407:78-84, 1997.

64. Hermonat, P. L. et al., J. Virology 51:329-339, 1984.

65. Ignarro, L. J. and C. Colombo. Science 180:1181-1183, 1973.

66. Ilhan, M. and I. Sahin. Eur. J. Pharmacol. 131:293-296, 1986.

67. Ing, N. H. etal., J. Biol. Chem. 267:17617-17623, 1992.

68. Isawa, T. et al., Amer. Rev. Respir. Dis. 141:715-720, 1990.

69. Jaccoby, S. et al., Gen. Comp. Endocrinol. 106:327-337, 1997.

70. Jiang, L. and G. Kunos, Gene 163:331-332, 1995.

71. Jiang, L. et al, Biochemistry 35:13136-13146, 1996.

72. Kelsen, S. G. et al., Amer. J. Resp. Crit. Care Med. 152:1774-1783, 1995.

73. Kobilka, B. K. et al., J. Biol. Chem. 262:7321-7327, 1987.

74. Koenig, J. A. and J. M. Edwardson. Trends Pharmacol. Sci. 18:276-287, 1997.

75. Koyama, S. S. I. et al., Amer. J. Physiol. 257:L130-L136, 1989.

76. LaFace, D. et al., Virology 162:483-486, 1988.

77. Landt, O. et al., Am. Rev. Respir. Dis. 139:552-555, 1989.

79. Lukert, B. P. and L. G. Raisz, Ann. Intern. Med. 112:352-364, 1990.

80. Majors, J. and H. E. Varmus. Proc. Natl.Acad. Sci. USA 80:5866-5870, 1983.

81. Mak, J. C. W. et al.,J. Clin. Invest. 96: 99-106, 1995.

82. Malbon, C. C. and J. R. Hadcock. Biochem. Biophys. Res. Comm. 154:676-681, 1988.

83. Mangelsdorf, D. J. et al., Cell 83:835-839, 1995.

84. Mano, K. et al., Life Sci. 25:1925-1930, 1979.

85. Mastrangeli, A. et al., J. Clin. Invest. 91:225-234, 1993.

86. McGehee, R. E., Jr. et al., Molec. Cell. Endocrinol. 74:1-9, 1990.

87. McGraw, D. W. et al., Exp. Lung Res. 21:535-546, 1995.

88. McGraw, D. W. et al., Biochim. Biophys. Acta 1305:135-138, 1996.

89. McGraw, D. W. et al., Amer. J. Resp. Crit. Care Med. 157:A745, 1998.

90. Minneman, K. P. et al., Biochem. Soc. Trans. 21:1109-1114, 1993.

91. Mittal, R., et al.,. Molec. Endocrinol. 8: 1701-1708, 1994.

92. Morrison, K. J., et al., Life Sci. 52:2123-2130, 1993.

93. Mortensen, J. et al., Thorax 46:817-823, 1991.

94. Muzyczka, N. Curr. Top. Microbiol. Immunol. 158:97-129, 1992.

95. Nakada, M. T. et al., Biochem. J. 260:53-59, 1989.

96. Nassif, E. et al., J. Allergy Clin. Immunol. 80:518-529, 1987.

97. Nelson, H. S. New Engl. J. Med. 333:499-506, 1995.

98. Nijkamp, F. P. Life Sci. 52:2073-2082, 1993.

99. Nijkamp, F. P. et al., Physiol. Rev. 72:323-367, 1992.

100. Nimar, H. L. et al., Proc. Natl. Sci. USA 80:4949-4953, 1983.

101. Nogami, M. et al., Clin. Sci. 85:651-657, 1993.

102. Nordeen, S. K., BioTechniques 6:454-457, 1988.

103. Nordeen, S. K. et al., Molec. Endocrinol. 4:1866-1873, 1990.

104. Norris, J. S. et al., Molec. Cell. Biochem. 74:21-27, 1987.

105. Ohuchi, K. et al., Immunopharmacol. 20:81-88, 1990.

106. Ota, A. et al., Neurosci. Lett. 160:96-100, 1993.

107. Otake, K. et al., Human Gene Therap. 9:2207-2222, 1998.

108. Oudrhiri, N. et al., Proc. Natl. Acad. Sci. USA 94:1651-1656, 1997.

109. Peachell, P. T. et al., J. Immunol. 140:571-579, 1988.

110. Pearce, N. et al., Thorax 45:170-175, 1990.

111. Persson, C. G. A. Life Sci. 52:2111-2121, 1993.

112. Plopper, C. G. et al., Exp. Lung Res. 5:281-294, 1983.

113. Pratt, W. B. J. Biol. Chem. 268:21455-21458, 1993.

114. Qing, K. et al., J. Virol. 72:1593-1599, 1998.

115. Rasmussen, H. et al., Am. J. Physiol. 258:L279-L288, 1990.

116. Rennard, S. I. et al, The Lung: Scientific Foundations, (R. G. Crystal and J. B. West, eds.), Raven Press, Ltd., New York, pp. 157-167, 1991.

117. Renzi, P. M. et al., Amer. J. Respir. Cell Mol. Biol. 15:367-373, 1996.

118. Robbins, R. A. et al., Am. J. Physiol. 257:109-L115, 1989.

119. Ron, D. et al., Molec. Cell. Biol. 10:4389-4395, 1990.

120. Rooklin, A. R. et al., J. Allergy Clin. Immunol. 63:383-386, 1979.

121. Salonen, R. O. Acta Pharmacol. et Toxicol. 57: 147-153, 1985.

122. Samulski, R. J. et al., EMBO J. 10:3941-3950, 1991.

123. Sanger, F. et al., Proc. Natl. Acad. Sci. USA 74: 5463-5467, 1977.

124. Scheidereit, C. et al., Nature 304:749-752, 1983.

125. Schell, D. N. et al., Amer. J. Respir. Cell. Mol. Biol. 7:317-324, 1992.

126. Schule, R. et al., Science 242:1418-1420, 1988.

127. Schule, R. et al., Nature 332:87-90, 1988.

128. Sears, M. R. et al., Lancet 336:1391-1396, 1990.

129. Shapiro, G. G. et al., J. Allergy Clin. Immunol. 57:430-439, 1976.

130. Shenfield, G. M. et al., Thorax 30:430-435, 1975.

131. Sime, P. J. et al., J. Clin. Invest. 100:768-776, 1997.

132. Spahn, J. D. et al., Asthma (P. J. Barnes et al., eds.), Lippincott-Raven Publishers, Philadelphia, pp. 1619-1638, 1997.

133. Spitzer, W. O. et al., New Engl. J. Med. 326:560-561, 1992.

134. Steggles, A. W. BioTechniques 7: 241-242, 1989.

135. Stellato, C. et al., Asthma (P. J. Barnes et al., eds.), Lippincott Raven Publishers, Philadelphia, pp. 1569-1596, 1997.

136. Strader, C. D. et al., FASEB J. 9:745-754, 1995.

137. Suzuki, M. et al., Human Gene Therapy 7:1883-1893, 1996.

138. Svedmyr, N. Amer. Rev. Respir. Dis. 141: S31-S38, 1990.

139. Szarek, J. L. et al., J. Appl. Physiol. 78:1679-1687, 1995.

140. Takahashi, H. and H. Iizuka. Brit. J. Dermatol. 124:341-347, 1991.

141. Taylor, D. R. et al., Med. Clin. North Amer. 80:719-748, 1996.

142. Taylor, D. R. et al., Thorax 48:134-138, 1993.

143. Tinkelman, D. G. et al., Pediatrics 92:64-77, 1993.

144. Tolstoshev, P. The Molecular Basis of Cancer. (J. Mendelsohn et al., eds.), W. B. Saunders Co., Philadelphia, pp. 534-557, 1995.

145. Toogood, J. H. et al., J. Allergy Clin. Immunol. 91:571-579, 1993.

146. Trapnell, B. C. Adv. Drug Del. Rev. 12:185-199, 1994.

147. von Zastrow, M. et al., J. Biol. Chem. 268:763-766, 1993.

148. Wang, J. -C. et al., Molec. Endocrinol. 10: 794-800, 1996.

149. Watanabe, A. et al., Amer. J. Resp. Crit. Care Med. 152:64-70, 1995.

150. Wilson, J. M. J. Clin. Invest. 96:2547-2554, 1995

151. Woolcock, A. J, Am. J. Resp. Crit. Care Med. 154:545-548, 1996.

152. Wright, A. P. et al., J. Steroid Biochem. Molec. Biol. 47:11-19, 1993.

153. Xing, Z. et al., J. Clin. Invest. 97:1102-1110, 1996.

154. Yamamoto, K. R. Harvey Lect. 91:1-19, 1997.

155. Yu, X. Y. et al., Am. J. Physiol. 262:L229-L234, 1992.

156. Yukawa, T. D. et al., Am. Rev. Respir. Dis. 141:1446-1452, 1990.

157. Zhong, H. and K. P. Minneman. Molec. Pharmacol. 44:1085-1093, 1993.

158. Zolotukhin, S. et al., J. Virolol. 70:4646-4654, 1996.

159. C. P. Hodgson, Bio/Technology, 13, 222-225 1995.

160. Kay et al., Proc. Natl. Acad. Sci. USA, 94:12744 1997.

161. Stone et al., J. Endocrinol. 164:103-118, 2000.

162. Samulski et al., J. Virol. 63:3822-3828, 1989.

163. Kotin and Berns, Virology 170: 460-467,1989.

164. Summerford et al., Nature Medicine 5:78-82, 1999.

165. Xiao et al., J. Virol. 72:2224-2232, 1989.

166. Demoly et al., Gene Therapy, 4:507, 1997.

167. Rogers et al. in Thorax 53: 200, 1998.

168. McGraw et al., AJP 279:L379-389, 2000.

169. McGraw et al. J. Biol. Chem. 274:32241-32247, 1999.

170. Drazner et al., J. Clin. Invest., 99: 288 1997

171. Kobilka et al., Nature 329:75 1986.

172. Xiao, X. et al., J. Virology 70:8098-8108, 1996.

173. Bouvier et al., Nature 333:370-373,1988.

174. Krupnick et al., Annual Review of Pharmacology and Toxicology 38:289-319, 1998

175. Samama et al. Journal of Biological Chemistry 268:4625-4636, 1993.

176. Burcin et al., Proc. Natl. Acad. Sci. USA 96: 355-360, 1999.

177. Rounds et al., Textbook of Pulmonary Diseases, 6^{th} Ed.pp. 1273-1284, Baum et al. Eds., Lippincott-Raven Publishers, Philadelphia, Pa., 1998.

178. Fullerton et al., Surgery 119 (1): 98-103, 1996.

179. Sundeep, Chest 115(6): 1708-1719, 1999.

We claim

1. A method for providing a β₂-adrenergic receptor (EAR) to airway epithelial cells, airway smooth muscle cells, blood vessel endothelial cells, blood vessel smooth muscle cells or a combination thereof, of a human subject comprising:
administering to at least one cell type selected from the group consisting of airway epithelial cells, airway smooth muscle cells, blood vessel endothelial cells, and blood vessel smooth muscle cells of a human subject, a first composition comprising a vector comprising a DNA sequence encoding a β₂AR operably linked to a promoter that is functional in at least one of said cells of said subject, under conditions whereby the DNA sequence encoding said β₂AR is expressed in at least one of said cells.

2. The method of claim 1, wherein said DNA sequence encodes a β₂AR that is modified as compared to the native β₂AR.

3. The method of claim 1, wherein said promoter is an inducible promoter, and said method further comprises:
administering a second composition comprising a hormone or pharmacological agent that induces said promoter to express said β₂AR in at least one of said cells.

4. The method of claim 1, wherein said method further comprises:
administering a second composition comprising at least one β₂-adrenergic agonist to said cells of said subject.

5. The method of claim 4, wherein said promoter is an inducible promoter, said method further comprises:
administering a third composition comprising a hormone or pharmacological agent that induces said promoter to express said β₂AR in at least one of said cells.

6. A host cell transfected by a vector comprising a DNA sequence encoding a β₂AR operably linked to a promoter that is functional in said cell.

7. The method of claim 6, wherein said DNA sequence encodes a β₂AR that is modified as compared to the native β₂AR.

8. A method of treating a human subject having airway or vascular disease comprising:
administering to at least one cell type selected from the group consisting of airway epithelial cells, airway smooth muscle cells, blood vessel endothelial cells, and blood vessel smooth muscle cells, a first composition comprising a vector comprising a DNA sequence encoding a β₂AR operably linked to a promoter that is functional in at least one of said cells of said subject, under conditions whereby the DNA sequence encoding said EAR is expressed in at least one of said cells; and
administering a second composition comprising at least one β₂-adrenergic agonist into said cells of said subject.

9. The method of claim 1 wherein said epithelial cell is an airway epithelial cell.

10. The method of claim 1, wherein said vector is a viral vector or a non-viral vector.

11. The method of claim 10, wherein said viral vector is selected from the group consisting of an adeno-associated vector (AAV), an adenovirus vector and a retrovirus vector.

12. The method of claim 10, wherein said non-viral vector is a liposome.

13. The method of claim 10, wherein said promoter is selected from the group consisting of a viral vector promoter and a mammalian cell specific promoter.

14. The method of claim 13, wherein said mammalian cell specific promoter is selected from the group consisting of an epithelial cell specific promoter, an endothelial cell specific promoter and a smooth muscle cell specific promoter.

15. The method of claim 13, wherein said viral vector promoter is a cytomegalovirus (CMV) promoter or an adeno-associated vector (AAV) promoter.

16. The method of claim 15, wherein said vector is an AAV vector and said promoter is a CMV promoter.

17. The method of claim 13, wherein said promoter is an inducible promoter.

18. The method of claim 17, wherein said method further comprises:
administering a composition comprising a hormone or pharmacological agent that induces said promoter to express said β₂AR in at least one of said cells.

19. The method of claim 1, wherein said vector further comprises at least one enhancer element or regulatory element.

20. The method of claim 1, wherein said first composition further comprises a pharmaceutically acceptable carrier for aerosol delivery or for intravenous delivery.

21. The method of claim 4, wherein said second composition is administered Isequentially after the administration of said first composition.

22. The method of claim 8, wherein said second composition is administered sequentially after the administration of said first composition.

23. The method of claim 3, wherein said first and second compositions further comprise a pharmaceutically acceptable carrier for aerosol delivery.

24. The method of claim 4, wherein said first and second compositions further comprise a pharmaceutically acceptable carrier for aerosol delivery.

25. The method of claim 5, wherein said first, said second and said third compositions further comprise a pharmaceutically acceptable carrier for aerosol delivery.

26. The method of claim 8, wherein said first and second compositions further comprise a pharmaceutically acceptable carrier for aerosol delivery.

27. The method of claim 8, wherein said DNA sequence encodes a β₂AR that is modified as compared to the native β₂AR.

28. The method of claim 2, wherein said modified β₂AR possesses at least one property selected from the group consisting of increased responsiveness to β₂AR agonists, increased affinity to β₂-adrenergic agonists, and capability to increase the potency of β₂AR agonists to stimulate downstream signal transduction pathways, as compared to the native β₂AR.

29. The method of claim 28, wherein said modified β₂AR is modified from the native β₂AR by the deletion of amino acids, substitution of amino acids, replacement of amino acids or a combination thereof.

30. A pharmaceutical composition comprising a vector comprising a DNA sequence encoding a β₂AR operably linked to a promoter that is functional in at least one cell of the airways or blood vessels of a human subject, wherein said cell is selected from the group consisting of an airway epithelial cells, airway smooth muscle cells, blood vessel endothelial cells and blood vessel smooth muscle cells; and a pharmaceutically acceptable carrier.

31. The pharmaceutical composition of claim 30, wherein said DNA sequence encodes a β₂AR that is modified as compared to the native β₂AR.

32. The pharmaceutical composition of claim 30, wherein said pharmaceutical composition is suitable for aerosol delivery or intravenous delivery.

33. A kit for the treatment of a human subject having airway or vascular disease comprising:
a first pharmaceutical composition comprising a vector comprising a DNA sequence encoding a β₂AR operably linked to a promoter that is functional in at least one cell of the airways or blood vessels of a human subject, wherein said cell is selected from the group consisting of an airway epithelial cells, airway smooth muscle cells, blood vessel endothelial cells and blood vessel smooth muscle cells; and a pharmaceutically acceptable carrier; and
a second pharmaceutical composition comprising at least one β₂-adrenergic agonist and a pharmaceutically acceptable carrier.

34. The kit of claim 33, wherein said β₂AR is modified as compared to the native β₂AR.

35. The kit of claim 33, wherein said promoter is an inducible promoter, said kit further comprises:
a third pharmaceutical composition comprising a hormone or pharmacological agent that induces said promoter to express said β₂AR in at least one of said cells.

36. The kit of claim 33, wherein said kit is for the treatment of said subject with an airway disease and said pharmaceutically acceptable carrier of said first and said second pharmaceutical composition are suitable for aerosol delivery.

37. The kit of claim 33, wherein said kit is for the treatment of said subjsect with a vascular disease and said pharmaceutically acceptable carriers of said first and second pharmaceutical composition are suitable for intravenous delivery.

38. A kit for the treatment of a human subject having airway or vascular disease comprising:
a first pharmaceutical composition comprising a vector comprising a DNA sequence encoding a β₂AR operably linked to a promoter that is functional in at least one cell of the airways or blood vessels of a human subject, wherein said cell is selected from the group consisting of an airway epithelial cells, airway smooth muscle cells, blood vessel endothelial cells and blood vessel smooth muscle cells; and a pharmaceutically acceptable carrier; and
a second pharmaceutical composition comprising a hormone or pharmacological agent that induces said promoter to express said β₂AR in at least one of said cells.

39. A kit for the treatment of a human subject having airway or vascular disease comprising:
a first pharmaceutical composition comprising at least one β₂-adrenergic agonist and a pharmaceutically acceptable carrier; and
a second pharmaceutical composition comprising a hormone or pharmacological agent that induces a promoter that is functional in at least one cell of the airways or blood vessels of a human subject, wherein said cell is selected from the group consisting of an airway epithelial cells, airway smooth muscle cells, blood vessel endothelial cells and blood vessel smooth muscle cells, said promoter to direct the expression of a β₂AR in at least one of said cells.

40. An in vitro method of expressing the β₂AR gene comprising:
transducing at least one mammalian cell with a recombinant vector that carries the nucleic acid sequence encoding the native β₂AR or a modified β₂AR, and expressing said β₂AR gene in said cell.

41. The method of claim 40, wherein said method further comprises
contacting said transduced cell with a pharmacological compound and evaluating the effect of the compound on the expression of said β₂AR gene.

42. The method of claim 41, wherein said mammalian cell is an epithelial cell.

43. The method of claim 41, wherein said vector is an AAV vector.
